(19) United States
(12) Patent Application Publication
(10) Pub. No.: US 2013/0267500 A1
(43) Pub. Date: Oct. 10, 2013

(54) 5-HT2C RECEPTOR AGONISTS IN THE TREATMENT OF DISORDERS AMELIORATED BY REDUCTION OF NOREPINEPHRINE LEVEL

(71) Applicants: Christen M. Anderson, Encinitas, CA (US); William R. Shanahan, Del Mar, CA (US)

(72) Inventors: Christen M. Anderson, Encinitas, CA (US); William R. Shanahan, Del Mar, CA (US)

(73) Assignee: Arena Pharmaceuticals, Inc.

(21) Appl. No.: 13/820,073

(22) PCT Filed: Aug. 31, 2011
(86) PCT No.: PCT/US2011/049955

§ 371 Date: Jun. 19, 2013

Related U.S. Application Data
(60) Provisional application No. 61/402,565, filed on Sep. 1, 2010.
(60) Provisional application No. 61/403,185, filed on Sep. 10, 2010.

(51) Int. Cl. A61K 31/55

(52) U.S. Cl. CPC A61K 31/55
USPC 514/217.01

(57) ABSTRACT

Uses of 5-HT_{2C} receptor agonists in the treatment of disorders ameliorated by reduction of an individual's norepinephrine level, wherein said disorders include but are not limited to hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

### FIELD

Provided are uses of 5-HT_{2C} receptor agonists in the treatment of disorders ameliorated by reduction of an individual's norepinephrine level, wherein said disorders include but are not limited to hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma. Said 5-HT_{2C} receptor agonists include but are not limited to lorcaserin.

### BACKGROUND

Norepinephrine (noradrenalin) is a neurotransmitter released in the brain as well as a hormone released peripherally by the adrenal glands into the blood. In some physiological contexts including but not limited to stress, obesity, and pheochromocytoma norepinephrine is elevated. Norepinephrine level can be increased by cellular release and can be inhibited by cellular reuptake and by catabolism. Elevation of norepinephrine can lead to disorders including but not limited to insulin resistance, cardiac hypertrophy, elevated heart rate, vasoconstriction, and hypertension, hyperglycemia, type 2 diabetes, hyperinsulinemia, heart failure, cardiomyopathy, cardiomyocyte hypertrophy in post-myocardial infarction remodeling, and acute pulmonary vasoconstriction. Kvetnansky R., et al., Catecholaminergic Systems: Structural and Molecular Genetic Approaches, Physiol. Rev. (2009) 89:535-606; Straznicky N. E., et al., Neuroadrenergic dysfunction in obesity: an overview of the effects of weight loss, Curr. Opin. Lipidol. (2010) 21:21-30; Bonisch H., et al., The Norepinephrine Transporter in Physiology and Disease, Handbook of Experimental Pharmacology (2006) 175:485-524. Compounds that lower norepinephrine levels, are useful in treating these diseases and conditions.

Dopamine is converted to norepinephrine by dopamine-β-hydroxylase (DBH). Inhibitors of dopamine-beta-hydroxylase (DBH) are reported (WO2009/097416) to be useful for a variety of clinical purposes including but not limited to regulation of lipid metabolism, vasodilation, and treatment of hypertension, congestive heart failure, hyperthyroidism, Parkinson disease, post-traumatic stress disorder, and reward deficiency syndrome (RDS), and other diseases or conditions which are positively affected by increased dopamine and/or by decreased norepinephrine. Compounds that lower norepinephrine levels, are also useful in treating these diseases and conditions.

The 5-HT₂ subfamily of serotonin (5-hydroxytryptamine; 5-HT) receptors contains three highly homologous receptor subtypes: 5-HT_{2A}, 5-HT_{2B}, and 5-HT_{2C}. The human 5-HT_{2C} receptor is predominantly expressed in brain. Pasqualetti M., et al., Distribution and Cellular Localization of the Serotonin Type 2C Receptor Messenger RNA in Human Brain, Neuroscience (1999) 92:601-611;

Agonists of the 5-HT_{2C} receptor have been shown to be useful for obesity and weight management. Liu K K-C., et al., Orally active and brain permeable proline amides as highly selective 5HT_{2C} agonists for the treatment of obesity, Bioorg. Med. Chem. Lett. (2010) 20:2365-2369; Smith S. R., et al., Lorcaserin (ADP356), a Selective 5-HT_{2C} Agonist, Reduces Body Weight in Obese Men and Women, Obesity (2008) 17:494-503; Thomsen W. J., et al., Lorcaserin, a Novel Selective Human 5-Hydroxytryptamine-2C Agonist: in Vitro and in Vivo Pharmacological Characterization, Journal of Pharmacology and Experimental Therapeutics (2008) 325:577-587; Shimada I, et al., Synthesis and structure-activity relationships of a series of substituted 2-(1H-furo[2,3-g]indazol-1-yl)ethylamine derivatives as 5-HT_{2C} receptor agonists, Bioorg. Med. Chem. (2008) 16:1966-1982; Smith B. M., et al., The potential use of selective 5-HT_{2C} agonists in treating obesity, Expert Opin. Investig. Drugs (2006) 15:257-266; Nilsson B. M., 5-Hydroxytryptamine 2C (5-HT_{2C}) Receptor Agonists as Potential Antiobesity Agents, J. Med. Chem. (2006) 49:4023-4034; Rosenzweig-Lipson S., et al., Antiobesity-like effects of the 5-HT_{2C} receptor agonist WAY-161503, Brain Res. (2006) 1073-1074:240-251; Jandacek R. J., APD-356 Arena, Cur. Opin. Investig. Drugs (2005) 6:1051-1056.

(R)-8-Chloro-1-methyl-2,3,4,5-tetrahydro-1H-3-benzazepine (Compound 1) is an exemplary potent, selective 5-HT_{2C} receptor agonist.

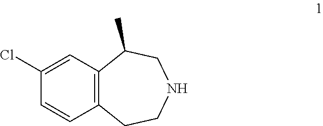

Compound 1 is disclosed in PCT patent publication WO2003/086303, which is incorporated herein by reference in its entirety. Various synthetic routes to (R)-8-chloro-1-methyl-2,3,4,5-tetrahydro-1H-3-benzazepine, its related salts, enantiomers, crystalline forms, and intermediates, have been reported in PCT publications, WO 2005/019179, WO 2006/069363, WO 2007/120517, WO 2008/070111, WO 2009/111004, and in U.S. provisional application 61/396,752 each of which is incorporated herein by reference in its entirety.

Combinations of (R)-8-chloro-1-methyl-2,3,4,5-tetrahydro-1H-3-benzazepine with other agents, including without limitation, phentermine, and uses of such combinations in therapy are described in WO 2006/071740, which is incorporated herein by reference in its entirety.

The following United States provisional applications are related to (R)-8-chloro-1-methyl-2,3,4,5-tetrahydro-1H-3-benzazepine: 61/402,578; 61/403,143; 61/402,580; 61/402,628; 61/403,149; 61/402,589; 61/402,611; 61/402,565; 61/403,185; each of which is incorporated herein by reference in its entirety.

The following applications are related to (R)-8-chloro-1-methyl-2,3,4,5-tetrahydro-1H-3-benzazepine and have the same filing date as the subject application: Attorney Reference Number 178.WO1, a PCT application which claims priority to U.S. provisional applications 61/402,578 and 61/403,143; Attorney Reference Number 181.WO1, a PCT application which claims priority to U.S. provisional application 61/402,580; Attorney Reference Number 186.WO1, a PCT application which claims priority to U.S. provisional applications 61/402,628 and 61/403,149; Attorney Reference Number 187.WO1, a PCT application which claims priority to U.S. provisional application 61/402,589; and Attorney Reference Number 188.WO1, a PCT application which claims priority to U.S. provisional application 61/402,611; each of which is incorporated herein by reference in its entirety.

(R)-8-Chloro-1-methyl-2,3,4,5-tetrahydro-1H-3-benzazepine hydrochloride (lorcaserin hydrochloride) is an agonist of the 5-HT_{2C} receptor and shows effectiveness at reducing obesity in animal models and humans. In December 2009, Arena Pharmaceuticals, Inc. submitted a New Drug Application, or NDA, for lorcaserin to the FDA. The NDA submission is based on an extensive data package from lorcaserin's clinical development program that includes 18 clinical trials totaling 8,576 patients. The pivotal phase 3 clinical trial program evaluated nearly 7,200 patients treated for up to two years, and showed that lorcaserin consistently produced significant weight loss with excellent tolerability. About two-thirds of patients achieved at least 5% weight loss and over one-third achieved at least 10% weight loss. On average, patients lost 17 to 18 pounds or about 8% of their weight. Secondary endpoints, including body composition, lipids, cardiovascular risk factors and glycemic parameters improved compared to placebo. In addition, heart rate and blood pressure went down. Lorcaserin did not increase the risk of cardiac valvulopathy. Lorcaserin improved quality of life, and there was no signal for depression or suicidal ideation. The only adverse event that exceeded the placebo rate by 5% was generally mild or moderate, transient headache. Based on a normal BMI of 25, patients in the first phase 3 trial lost about one-third of their excess body weight. The average weight loss was 35 pounds or 16% of body weight for the top quartile of patients in the second phase 3 trial.

The present invention relates to the surprising and unexpected discovery by Applicant that administering the selective 5-HT_{2C} receptor agonist (R)-8-chloro-1-methyl-2,3,4,5-tetrahydro-1H-3-benzazepine to an individual causes a reduction of the individual's norepinephrine level independently of weight-loss.

### SUMMARY

The present invention pertains to methods relating to screening assays performed with the 5-HT_{2C} receptor for identifying 5-HT_{2C} receptor agonists, use of the 5-HT_{2C} receptor for identifying compounds useful for treating or preventing a condition ameliorated by reduction of norepinephrine level in an individual, methods for using 5-HT_{2C} receptor agonists in model systems for determining efficacy or usefulness, methods for using 5-HT_{2C} receptor agonists in the treatment or prevention of a condition ameliorated by reduction of norepinephrine level in an individual. In certain embodiments, the individual is a human.

One of the objectives of the present disclosure is to allow the skilled artisan to identify 5-HT_{2C} receptor agonists that are appropriate for treating disorders ameliorated by the reduction of an individual's norepinephrine level. Another objective of the present disclosure is to allow the skilled artisan who has identified a series of 5-HT_{2C} receptor agonists that are appropriate for treating disorders ameliorated by the reduction of an individual's norepinephrine level, to subsequently optimize that series through chemical modifications to improve biological and physiochemical properties.

One aspect of the present invention pertains to methods for reducing a concentration of norepinephrine in an individual, comprising administering to the individual, by the individual or by a caregiver, one or more doses of a medicament comprising a therapeutically effective amount of a 5-HT_{2C} receptor agonist.

One aspect of the present invention pertains to methods for maintaining a reduced concentration of norepinepherine in an individual, comprising administering to the individual, by the individual or by a caregiver, one or more doses of a medicament comprising a therapeutically effective amount of a 5-HT_{2C} receptor agonist.

One aspect of the present invention pertains to methods for manufacturing a medicament for reducing a concentration of norepinephrine in an individual, comprising admixing a 5-HT_{2C} receptor agonist with at least one pharmaceutically acceptable excipient; wherein the 5-HT_{2C} receptor agonist has been administered to a mammal in whom a norepinephrine concentration has been measured.

One aspect of the present invention pertains to methods for manufacturing a medicament for reducing a concentration of norepinephrine in an individual, comprising: measuring a norepinephrine concentration in a mammal; and admixing a 5-HT_{2C} receptor agonist with at least one pharmaceutically acceptable excipient; wherein said 5-HT_{2C} receptor agonist has been administered to said mammal.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in the mammal; and admixing the 5-HT_{2C} receptor agonist with at least one pharmaceutically acceptable excipient.

One aspect of the present invention pertains to methods for treating a disorder ameliorated by a reduction of a concentration of norepinephrine in an individual, comprising admixing a 5-HT_{2C} receptor agonist with at least one pharmaceutically acceptable excipient; wherein said 5-HT_{2C} receptor agonist has been administered to a mammal in whom a norepinephrine concentration has been measured.

One aspect of the present invention pertains to methods for manufacturing a medicament for treating a disorder ameliorated by a reduction of a concentration of norepinephrine in an individual, comprising: measuring a norepinephrine concentration in a mammal; and admixing a 5-HT_{2C} receptor agonist with at least one pharmaceutically acceptable excipient; wherein said 5-HT_{2C} receptor agonist has been administered to said mammal.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in said mammal; and admixing said 5-HT_{2C} receptor agonist with at least one pharmaceutically acceptable excipient.

One aspect of the present invention pertains to methods for manufacturing a medicament for reducing a concentration of norepinephrine in an individual, comprising resynthesizing a 5-HT_{2C} receptor agonist; wherein said 5-HT_{2C} receptor agonist has been administered to a mammal in whom a norepinephrine concentration has been measured.

One aspect of the present invention pertains to methods for manufacturing a medicament for reducing a concentration of norepinephrine in an individual, comprising: measuring a norepinephrine concentration in a mammal; and resynthesizing a 5-HT_{2C} receptor agonist; wherein said 5-HT_{2C} receptor agonist has been administered to said mammal.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in said mammal; and resynthesizing said 5-HT_{2C} receptor agonist.

One aspect of the present invention pertains to methods for manufacturing a medicament for treating a disorder ameliorated by a reduction of a concentration of norepinephrine in an individual, comprising resynthesizing a 5-HT_{2C} receptor agonist; wherein said 5-HT_{2C} receptor agonist has been administered to a mammal in whom a norepinephrine concentration has been measured.

One aspect of the present invention pertains to methods for manufacturing a medicament for treating a disorder ameliorated by a reduction of a concentration of norepinephrine in an individual, comprising: measuring a norepinephrine concentration in a mammal; and resynthesizing a 5-HT_{2C} receptor agonist; wherein said 5-HT_{2C} receptor agonist has been administered to said mammal.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in said mammal; and resynthesizing said 5-HT_{2C} receptor agonist.

One aspect of the present invention pertains to methods for treating a disorder ameliorated by reduction of a concentration of norepinephrine in an individual, comprising administering to the individual, by the individual or by a caregiver, one or more doses of a medicament comprising a therapeutically effective amount of a 5-HT_{2C} receptor agonist.

It is understood by one of ordinary skill in the art that any of the methods of the present invention optionally apply to a catecholamine other than norepinephrine, such as, epinephrine.

One aspect of the present invention pertains to 5-HT_{2C} receptor agonists for use by an individual or caregiver in reducing a concentration of norepinephrine in the individual.

One aspect of the present invention pertains to 5-HT_{2C} receptor agonists, for use by an individual or caregiver in reducing a concentration of norepinephrine in the individual and for maintaining the reduced concentration of norepinepherine.

One aspect of the present invention pertains to 5-HT_{2C} receptor agonists for use in treating a disorder ameliorated by reduction of a concentration of norepinephrine.

### BRIEF DESCRIPTION OF THE DRAWINGS

FIG. 1: Longitudinal analysis of 24-h total urine norepinepherine in humans at baseline, 7 days and 56 days post-treatment with lorcaserin or placebo.

FIG. 2: Longitudinal analysis of 24-h urine norepinepherine in humans at baseline, 7 days and 56 days post-treatment with lorcaserin or placebo.

FIG. 3: Longitudinal analysis of body weight in humans at baseline, 6 days and 56 days post-treatment with lorcaserin or placebo.

### DETAILED DESCRIPTION

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### DEFINITIONS

For clarity and consistency, the following definitions will be used throughout this patent document.

The term “5-HT_{2A} serotonin receptor” or “5-HT_{2A} receptor” as used herein includes human amino acid sequences found in GenBank accession number NP_{—}000612, and naturally-occurring allelic variants thereof, and mammalian orthologs thereof. A preferred human 5-HT_{2A} receptor for use in screening and testing of the compounds of the invention is provided by the nucleotide sequence and the corresponding amino acid sequence found in GenBank accession number NP_{—}000612.

The term “the 5-HT_{2B} serotonin receptor” or “5-HT_{2B} receptor” as used herein includes human amino acid sequences found in GenBank accession number NP_{—}000858, and naturally-occurring allelic variants thereof, and mammalian orthologs thereof. A preferred human 5-HT_{2B} receptor for use in screening and testing of the compounds of the invention is provided by the nucleotide sequence and the corresponding amino acid sequence found in GenBank accession number NP_{—}000858.

The term “5-HT₂ serotonin receptor” or “5-HT₂ receptor” as used herein includes human amino acid sequences found in GenBank accession number NP_{—}000859, and naturally-occurring allelic variants thereof, and mammalian orthologs thereof. A preferred human 5-HT₂ receptor for use in screening and testing of the compounds of the invention is provided by the nucleotide sequence and the corresponding amino acid sequence found in GenBank accession number NP_{—}000859.

The term “agonist” refers to a moiety that interacts with and activates a receptor, such as the 5-HT_{2C} serotonin receptor, and initiates a physiological or pharmacological response characteristic of that receptor.

The term “antagonist” refers to a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “baseline” refers to the measure of particular variable prior to the commencement of an experiment or a course of treatment. For example, the term “baseline” when used in reference to norepinephrine level in an individual may refer to the amount of norepinephrine in the individual's brain, plasma, blood or urine prior to the administration of a course of treatment comprising one or more doses of a 5-HT₂ agonist.

The term “individual” refers to both humans and non-human mammals. Non-human mammals include but are not limited to rodents such as mice and rats, etc. rabbits, dogs, cats, swine, cattle, sheep, horses, and non-human primates such as monkeys and apes, etc.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. In some embodiments, the baseline intracellular response is inhibited in the presence of the inverse agonist by at least 30%, as compared with the baseline response in the absence of the inverse agonist. In some embodiments, the baseline intracellular response is inhibited in the presence of the inverse agonist by at least 50%, as compared with the baseline response in the absence of the inverse agonist. In some embodiments, the baseline intracellular response is inhibited in the presence of the inverse agonist by at least 75%, as compared with the baseline response in the absence of the inverse agonist.

The term “mammal” refers to both humans and non-human mammals. Non-human mammals include but are not limited to rodents such as mice and rats, etc. rabbits, dogs, cats, swine, cattle, sheep, horses, and non-human primates such as monkeys and apes, etc.

The term “modulate or modulating” shall be taken to mean an increase or decrease in the amount, quality, response or effect of a particular activity, function or molecule.

The term “neutral antagonist” shall be taken to mean a moiety which blocks the affects of an agonist at the target receptor but does not significantly effect the level of constitutive receptor activity.

The term “inverse agonist” are agents which block the effects of an agonist at the target receptor and also suppress spontaneous receptor activity.

The term “orally bioavailable” as used herein refers to a compound which reaches an individual's systemic circulation unchanged following oral-administration of a medicament comprising the compound to the individual. In some embodiments, at least 10% of the compound reaches the systemic circulation unchanged following oral-administration. In some embodiments, at least 25% of the compound reaches the systemic circulation unchanged following oral-administration. In some embodiments, at least 50% of the compound reaches the systemic circulation unchanged following oral-administration. In some embodiments, at least 75% of the compound reaches the systemic circulation unchanged following oral-administration. In some embodiments, about 90% of the compound reaches the systemic circulation unchanged following oral-administration.

The term “partial agonist” is intended to mean a moiety that interacts with and activates a receptor, such as the 5-HT_{2C} serotonin receptor, and initiates a physiological or pharmacological response characteristic of that receptor but to a lesser degree/extent than full agonists.

The term “pharmaceutical composition” is intended to mean a composition comprising at least one active ingredient; including but not limited to, salts, solvates, and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “small molecule” shall be taken to mean a biologically-active organic compound with a molecular weight between about 100 g/mol and about 900 g/mol.

The term “substantial amount of weight” as used herein is intended to mean about 1% or more of an individual's baseline bodyweight. In some embodiments, a substantial amount of weight is intended to mean about 2% or more of an individual's baseline bodyweight. In some embodiments, a substantial amount of weight is intended to mean about 3% or more of an individual's baseline bodyweight. In some embodiments, a substantial amount of weight is intended to mean about 4% or more of an individual's baseline bodyweight.

The term “therapeutically effective amount” is intended to mean the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) Preventing the disease, for example, preventing a disease, condition or disorder in an individual that may be predisposed to the disease, condition or disorder but does not yet experience or display the pathology or symptomatology of the disease;

(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) Ameliorating the disease, for example, ameliorating a disease, condition or disorder in an individual that is experiencing or displaying the pathology or symptomatology of the disease, condition or disorder (i.e., reversing the pathology and/or symptomatology).

The term “treatment” as used herein refers to one or more of the following:

(1) Preventing the disease, for example, preventing a disease, condition or disorder in an individual that may be predisposed to the disease, condition or disorder but does not yet experience or display the pathology or symptomatology of the disease;

(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) Ameliorating the disease, for example, ameliorating a disease, condition or disorder in an individual that is experiencing or displaying the pathology or symptomatology of the disease, condition or disorder (i.e., reversing the pathology and/or symptomatology).

### 5-HT_{2C} Receptor Agonists

Serotonin (5-hydroxytryptamine, 5-HT) mediates a wide variety of central and peripheral psychological and physiological effects through fourteen mammalian 5-HT receptor subtypes, grouped into seven families 5-HT₁₋₇ (Sanders-Bush and Mayer, 2006). The 5-HT₂ family consists of the 5-HT_{2A}, 5-HT_{2B}, and 5-HT_{2C} membrane-bound G protein-coupled receptors (GPCRs) that signal primarily through Gαq to activate phospholipase (PL) C and formation of inositol phosphates (IP) and diacylglycerol (DAG) second messengers (Raymond et al., 2001). The human 5-HT_{2C} receptor (Saltzman et al., 1991) apparently is found exclusively in brain where it is widely expressed and putatively involved in several (patho)-physiological and psychological processes, including, ingestive behavior (Tecott et al., 1995), cocaine addiction (Fletcher et al., 2002; Rocha et al., 2002; Muller and Huston, 2006), sleep homeostasis (Frank et al., 2002), anxiety (Kennett et al., 1994; Sard et al., 2005; Heisler et al., 2007), depression (Tohda et al., 1989; Palvimaki et al., 1996), epilepsy (Heisler et al., 1998), Alzheimer's disease (Arjona et al., 2002; Stein et al., 2004), motor function (Heisler and Tecott, 2000; Segman et al., 2000), psychosis (Marquis et al., 2007; Siuciak et al., 2007) and response to antipsychotic drugs (Veenstra-VanderWeele et al., 2000; Reynolds et al., 2005). Thus, the importance of the 5-HT_{2C} receptor as a pharmacotherapeutic target has been apparent for over 10 years.

One challenge regarding drug discovery targeting the 5-HT_{2C} receptor is that this GPCR shares a transmembrane domain (TMD) sequence identity of about 80% with the 5-HT_{2A} receptor and about 70% with the 5-HT_{2B} receptor (Julius et al., 1988; 1990). The highly conserved TMDs and similar second messenger coupling has made development of agonist ligands selective for the 5-HT_{2C} receptor especially difficult. Nevertheless, there is compelling evidence that activation of 5-HT_{2C} receptors reduces food intake and leads to anti-obesity effects. For example, 5-HT_{2C} knockout mice demonstrate increased feeding and obesity, and, they are resistant to the anorectic effects of dexfenfluramine (Tecott et al., 1995; Vickers et al., 1999; 2001; Heisler et al., 2002). Fenfluramine now is banned, because, although people using the drug showed weight loss due to activation of brain 5-HT_{2C} receptors, fenfluramine also activates 5-HT_{2A} receptors that may lead to adverse psychiatric (hallucinogenic) effects (Nichols, 2004) and 5-HT_{2B} receptors which causes valvular heart disease (Connolly et al., 1997; Fitzgerald et al., 2000; Rothman et al., 2000; Roth, 2007) and pulmonary hypertension (Pouwels et al., 1990; Launay et al., 2002).

The pharmacotherapeutic relevance of the 5-HT_{2C} receptor has stimulated intense interest in the development of a selective 5-HT_{2C} agonist. See for example, WO2003/086306, WO2005/003096, WO2005/016902, WO2005/042490, WO2005/042491, WO2006/065600, and WO2006/065706, each of which is incorporated by reference in its entirety.

WO2008/156707 disclosed (1R,3S)-(−)-trans-1-phenyl-3-dimethylamino-1,2,3,4-tetrahydronaphthalene, an agonist at human 5-HT_{2C} receptors, and an antagonist at 5-HT_{2A} and 5-HT_{2B} receptors:

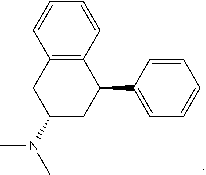 [SMILES: CN(C)[C@@H]1Cc2ccccc2[C@@H](c2ccccc2)C1]

Further exemplary 5-HT_{2C} agonists include the following: PNU 22394 (Hester et al., J. Med. Chem. (1968), 11(1), 101-6):

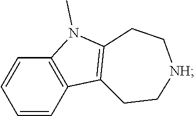

RO 60-0175 (U.S. Pat. No. 5,494,928):

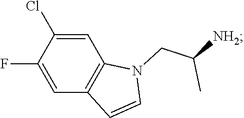

1-(5-fluorobenzofuran-7-yl)propan-2-amine (WO2000/044737):

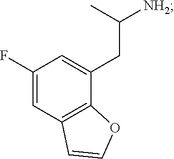

### ORG-37684 (WO 9943647):

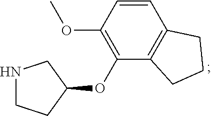 [SMILES: COc1ccc2c(c1O[C@H]1CCNC1)CCC2]

### MK-212 (Clineschmidt et al., European Journal of Pharmacology (1977), 44(1), 65-74):

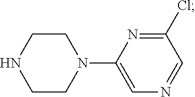 [SMILES: Clc1cncc(N2CCNCC2)n1]

(S)-1-(7-(methylthio)-2,3-dihydro-1H-pyrrolo[1,2-a]indol-9-yl)propan-2-amine (WO2000/012510):

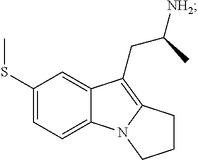 [SMILES: CSc1ccc2c(c1)c(C[C@H](C)N)c1n2CCC1]

### IK-264 (WO2000/077001):

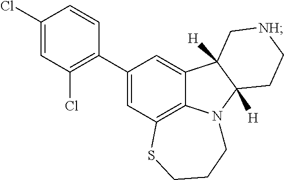 [SMILES: Clc1ccc(-c2cc3c4c(c2)[C@@H]2CNCC[C@@H]2N4CCCS3)c(Cl)c1]

(R)-7-chloro-1,2,3,4,10,10a-hexahydropyrazino[1,2-a]indole (WO2000/044753)

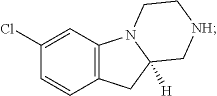 [SMILES: Clc1ccc2c(c1)N1CCNC[C@H]1C2]

### PNU-183933 (WO2000/076984):

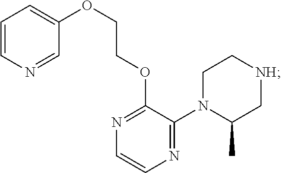 [SMILES: C[C@@H]1CNCCN1c1nccnc1OCCOc1cccnc1]

and

### WAY-161503 (WO2000/035922):

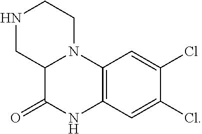 [SMILES: O=C1Nc2cc(Cl)c(Cl)cc2N2CCNCC12]

Further exemplary 5-HT_{2C} agonists include the 6-substituted 2,3,4,5-tetrahydro-1H-benzo[d]azepines disclosed in, WO2007/028131, WO2007/028082, WO2007/028083, WO2005/019180, and WO2005/082859, including 6-(2,2,2-trifluoroethylamino)-7-chloro-2,3,4,5-tetrahydro-1H-benzo[d]azepine, which is disclosed in WO2005/019180:

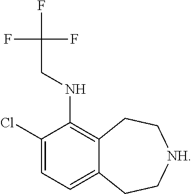 [SMILES: FC(F)(F)CNc1c(Cl)ccc2c1CCNCC2]

By way of further non-limiting example, 5-HT₂ receptor agonists have also been disclosed in, WO2011/097336, WO2011/071136, WO2011/019738, WO2011/016459, WO2011/005052, US2010/0317651, US2010/0298563, WO2010/129048, WO2010/060952, WO2010/038948, WO2009/128057, WO2009/079765, WO2009/063992, WO2009/061436, WO2009/051747, WO2009/037220, WO2008/117169, WO2008/052086, WO2008/052087, WO2008/052075, WO2008/052078, WO2008/010073, WO2008/009125, WO2008/007664, WO2007/140213, WO2007/132841, WO2007/084622, US2007/088022, WO2006/117304, WO2006/116151, WO2006/116221, US2006/241172, WO2006/077025, US2006/094752, US2006/089405, WO2006/044762, WO2006/01786, US2005/261347, WO2005/044812, WO2005/040146, WO2005/000849, US2004/186094, WO2004/056324, WO2004/000830, U.S. Pat. No. 6,667,303, WO2003/097636, WO2003/091257, WO2003/091251, WO2003/091250, WO2002/074746, WO2002/072584, WO2002/059129, WO2002/059127, U.S. Pat. No. 6,407,092, EP1213017, WO2002/040456, US2002/058689, US2002/055504, WO2002/036596, U.S. Pat. No. 6,372,745, US2001/051622, WO2001/068585, WO2000/076984, WO2000/044737, WO2000/035922, WO99/043647, and WO98/56768.

All combinations of the 5-HT₂ receptor agonists described herein are specifically embraced by the present invention just as if each and every combination was individually and explicitly recited. In addition, all subcombinations of the 5-HT₂ receptor agonists described herein as well as all subcombinations of uses and medical indications described herein, are also specifically embraced by the present invention just as if each and every subcombination of chemical groups and subcombination of uses and medical indications was individually and explicitly recited herein.

The ability of a compound to act as a 5-HT receptor agonist or antagonist can be determined using in vitro and in vivo assays that are known in the art. A functional assay measures a compound's biological activity in the assay system, whereas a binding assay measures a compound's affinity for a receptor. An assay based on the competition between a radio-labeled ligand and an unlabeled ligand in the reaction with a receptor is referred to as a competitive binding assay.

In vitro assays include assays that measure a compound's half maximal inhibitory concentration (IC₅₀). IC₅₀ is a measure of the effectiveness of a compound in inhibiting a response in a receptor. In vitro assays also include assays that measure the half maximal effective concentration (EC₅₀). EC₅₀ is a measure of the effectiveness of a compound in inducing a response in a receptor. One example of an in vitro assay that is used to measure the EC₅₀ of compounds at the 5-HT_{2A}, 5-HT_{2B}, and 5-HT_{2C} receptors is the intracellular IP3 accumulation assay described in Example 2.

One aspect of the present invention pertains to 5-HT_{2C} receptor agonists for use by an individual or caregiver in reducing a concentration of a catecholamine in the individual.

One aspect of the present invention pertains to 5-HT_{2C} receptor agonists for use by an individual or caregiver in reducing a concentration of epinephrine in the individual.

One aspect of the present invention pertains to 5-HT_{2C} receptor agonists for use by an individual or caregiver in reducing a concentration of norepinephrine in the individual.

In some embodiments, the reducing occurs within 56 days of administering the first dose.

In some embodiments, the reducing occurs within 7 days of administering the first dose.

In some embodiments, the reducing occurs between about 7 days and about 1 day of administering the first dose.

In some embodiments, the reducing occurs between about 56 days and about 1 day of administering the first dose.

In some embodiments, the reducing occurs between about 56 days and about 7 days of administering the first dose.

In some embodiments, the reducing is not dependent upon concomitant weight-loss in the individual.

In some embodiments, the individual does not lose a substantial amount of weight during the reducing.

In some embodiments, the individual loses a substantial amount of weight during the reducing and the reducing is greater than the amount of reducing expected by the individual or caregiver solely as a result of the individual loosing the substantial amount of weight.

In some embodiments, the concentration is a urine concentration.

In some embodiments, the concentration is a blood concentration.

In some embodiments, the concentration is a plasma concentration.

In some embodiments, the concentration is a brain concentration.

In some embodiments, the concentration is a cerebrospinal fluid concentration.

In some embodiments, the individual is hypernorepinephrinemic prior to the administering.

In some embodiments, the concentration of norepinephrine in the individual is at least 80 μg/24 h in urine prior to the administering.

In some embodiments, the concentration of norepinephrine in the individual is at least 600 pg/mL in blood prior to the administering.

In some embodiments, the reducing provides a reduced concentration of norepinephrine in the individual that is within the normal range.

In some embodiments, the reducing provides a reduced concentration of norepinephrine in the individual that is between about 15 and about 80 μg/24 h in urine.

In some embodiments, the reducing provides a reduced concentration of norepinephrine in the individual that is between about 0 and about 600 pg/mL in blood.

In some embodiments, the reducing provides a reduced concentration of norepinephrine in the individual at least about 10% lower than baseline.

In some embodiments, the reducing provides a reduced concentration of norepinephrine in the individual at least about 20% lower than baseline.

In some embodiments, the reducing provides a reduced concentration of norepinephrine in the individual at least about 30% lower than baseline.

In some embodiments, the reducing provides a reduced concentration of norepinephrine in the individual at least about 40% lower than baseline.

In some embodiments, the reducing provides a reduced concentration of norepinephrine in the individual at least about 50% lower than baseline.

In some embodiments, the reducing provides a reduced concentration of norepinephrine in the individual between about 10% lower than baseline and about 60% lower than baseline.

In some embodiments, the reducing provides a reduced concentration of norepinephrine in the individual between about 10% lower than baseline and about 50% lower than baseline.

In some embodiments, the reducing provides a reduced concentration of norepinephrine in the individual between about 10% lower than baseline and about 40% lower than baseline.

In some embodiments, the reducing provides a reduced concentration of norepinephrine in the individual between about 10% lower than baseline and about 20% lower than baseline.

In some embodiments, the reducing provides a reduced concentration of norepinephrine in the individual between about 10% lower than baseline and about 20% lower than baseline.

In some embodiments, the reducing provides a reduced concentration of norepinephrine in the individual between about 20% lower than baseline and about 60% lower than baseline.

In some embodiments, the reducing provides a reduced concentration of norepinephrine in the individual between about 20% lower than baseline and about 50% lower than baseline.

In some embodiments, the reducing provides a reduced concentration of norepinephrine in the individual between about 20% lower than baseline and about 40% lower than baseline.

In some embodiments, the reducing provides a reduced concentration of norepinephrine in the individual between about 20% lower than baseline and about 30% lower than baseline.

In some embodiments, the reducing provides a reduced concentration of norepinephrine in the individual between about 30% lower than baseline and about 60% lower than baseline.

In some embodiments, the reducing provides a reduced concentration of norepinephrine in the individual between about 30% lower than baseline and about 50% lower than baseline.

In some embodiments, the reducing provides a reduced concentration of norepinephrine in the individual between about 30% lower than baseline and about 40% lower than baseline.

In some embodiments, the reducing provides a reduced concentration of norepinephrine in the individual between about 40% lower than baseline and about 60% lower than baseline.

In some embodiments, the reducing provides a reduced concentration of norepinephrine in the individual between about 40% lower than baseline and about 50% lower than baseline.

In some embodiments, the reducing provides a reduced concentration of norepinephrine in the individual between about 50% lower than baseline and about 60% lower than baseline.

One aspect of the present invention pertains to 5-HT_{2C} receptor agonists, for use by an individual or caregiver in reducing a concentration of norepinephrine in the individual and for maintaining the reduced concentration of norepinepherine.

In some embodiments, the reduced concentration is a urine concentration.

In some embodiments, the reduced concentration is a blood concentration.

In some embodiments, the reduced concentration is a plasma concentration.

In some embodiments, the reduced concentration is a brain concentration.

In some embodiments, the reduced concentration is a cerebrospinal fluid concentration.

One aspect of the present invention pertains to 5-HT_{2C} receptor agonists for use in treating a disorder ameliorated by reduction of a concentration of norepinephrine.

In some embodiments, the disorder is selected from: hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

In some embodiments, the disorder is hypernorepinephrinemia.

In some embodiments, the disorder is cardiomyopathy.

In some embodiments, the disorder is cardiac hypertrophy.

In some embodiments, the disorder is cardiomyocyte hypertrophy in post-myocardial infarction remodeling.

In some embodiments, the disorder is elevated heart rate.

In some embodiments, the disorder is vasoconstriction.

In some embodiments, the disorder is acute pulmonary vasoconstriction.

In some embodiments, the disorder is hypertension.

In some embodiments, the disorder is heart failure.

In some embodiments, the disorder is cardiac dysfunction after stroke.

In some embodiments, the disorder is cardiac arrhythmia.

In some embodiments, the disorder is metabolic syndrome.

In some embodiments, the disorder is abnormal lipid metabolism.

In some embodiments, the disorder is hyperthermia.

In some embodiments, the disorder is Cushing syndrome.

In some embodiments, the disorder is pheochromocytoma.

In some embodiments, the disorder is epilepsy.

In some embodiments, the disorder is obstructive sleep apnea.

In some embodiments, the disorder is insomnia.

In some embodiments, the disorder is glaucoma.

In some embodiments, the disorder is osteoarthritis.

In some embodiments, the disorder is rheumatoid arthritis.

In some embodiments, the disorder is asthma.

In some embodiments, the individual is a human.

In some embodiments, the 5-HT_{2C} receptor agonist has an EC₅₀ of less than about 10 μM at the 5-HT_{2C} receptor.

In some embodiments, the 5-HT_{2C} receptor agonist has an EC₅₀ of less than about 1 μM at the 5-HT_{2C} receptor.

In some embodiments, the 5-HT_{2C} receptor agonist has an EC₅₀ of less than about 100 nM at the 5-HT_{2C} receptor.

In some embodiments, the 5-HT_{2C} receptor agonist has an EC₅₀ of between about 10 μM and about 1 nM at the 5-HT_{2C} receptor.

In some embodiments, the 5-HT_{2C} receptor agonist has an EC₅₀ of between about 10 μM and about 100 nM at the 5-HT_{2C} receptor.

In some embodiments, the 5-HT_{2C} receptor agonist has an EC₅₀ of between about 10 μM and about 1 μM at the 5-HT_{2C} receptor.

In some embodiments, the 5-HT_{2C} receptor agonist has an EC₅₀ of between about 1 μM and about 1 nM at the 5-HT_{2C} receptor.

In some embodiments, the 5-HT_{2C} receptor agonist has an EC₅₀ of between about 1 μM and about 100 nM at the 5-HT_{2C} receptor.

In some embodiments, the 5-HT_{2C} receptor agonist has an EC₅₀ of between about 100 nM and about 1 nM at the 5-HT_{2C} receptor.

In some embodiments, the 5-HT_{2C} receptor is a human 5-HT_{2C} receptor.

In some embodiments, the 5-HT_{2C} receptor agonist is a selective 5-HT_{2C} receptor agonist.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 10:1 and about 10000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 10:1 and about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 10:1 and about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 100:1 and about 10000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 100:1 and about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is between about 1000:1 and about 10000:1.

In some embodiments, the 5-HT_{2A} receptor is a human 5-HT_{2A} receptor; and the 5-HT_{2C} receptor is a human 5-HT_{2C} receptor.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 10:1 and about 10000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 10:1 and about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 10:1 and about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 100:1 and about 10000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 100:1 and about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at between about 1000:1 and about 10000:1.

In some embodiments, the 5-HT_{2B} receptor is a human 5-HT_{2B} receptor; and the 5-HT_{2C} receptor is a human 5-HT_{2C} receptor.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 10:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 10:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 10:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 100:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 100:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 100:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 1000:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 1000:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 1000:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 1000:1, and the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 1000:1.

In some embodiments, the selective 5-HT_{2C} receptor agonist is a partial agonist, an antagonist, an inverse agonist, or a neutral antagonist of the 5-HT_{2A} receptor.

In some embodiments, the selective 5-HT_{2C} receptor agonist is a partial agonist, an antagonist, an inverse agonist, or a neutral antagonist of the 5-HT_{2B} receptor.

In some embodiments, the selective 5-HT_{2C} receptor agonist is a partial agonist, an antagonist, an inverse agonist, or a neutral antagonist of the 5-HT_{2A} receptor; and a partial agonist, an antagonist, an inverse agonist, or a neutral antagonist of the 5-HT_{2B} receptor.

In some embodiments, the 5-HT_{2A} receptor is a human 5-HT_{2A} receptor; the 5-HT_{2B} receptor is a human 5-HT_{2B} receptor; and the 5-HT_{2C} receptor is a human 5-HT_{2C} receptor.

In some embodiments, the 5-HT_{2C} receptor agonist is a small molecule.

In some embodiments, the 5-HT_{2C} receptor agonist is orally bioavailable.

In some embodiments, the selective 5-HT_{2C} receptor agonist is selected from 8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is selected from (R)-8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is selected from (R)-8-chloro-1-methyl-2,3,4,5-tetrahydro-1H-3-benzazepine hydrochloride salt, and pharmaceutically acceptable solvates and hydrates thereof.

In some embodiments, the selective 5-HT_{2C} receptor agonist is (R)-8-chloro-1-methyl-2,3,4,5-tetrahydro-1H-3-benzazepine hydrochloride salt hemihydrate.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 10:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 10:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 10:1 and about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 10:1 and about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 100:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 1001 and about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is between about 1000:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 10:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 10:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 10:1 and about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 10:1 and about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 100:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 100:1 and about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2B} receptor is between about 1000:1 and about 10000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 10:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 10:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 10:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 10:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 100:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 10:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 100:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 100:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 1000:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 1000:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 10:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 1000:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 100:1.

In some embodiments, the ratio of the binding affinity of the selective 5-HT_{2C} receptor agonist at the 5-HT_{2C} receptor to the binding affinity of the selective 5-HT_{2C} receptor agonist at the 5-HT_{2A} receptor is at least about 1000:1, and the ratio of the binding affinity of the selective 5-HT_{2C} receptor agonist at the 5-HT_{2C} receptor to the binding affinity of the selective 5-HT_{2C} receptor agonist at the 5-HT_{2B} receptor is at least about 1000:1.

In some embodiments, the 5-HT_{2C} receptor agonist is a 5-HT_{2A} receptor antagonist.

In some embodiments, the 5-HT_{2C} receptor agonist is a 5-HT_{2B} receptor antagonist.

In some embodiments, the 5-HT_{2C} receptor agonist is a 5-HT_{2A} receptor partial agonist.

In some embodiments, the 5-HT_{2C} receptor agonist is a 5-HT_{2B} receptor partial agonist.

In some embodiments, the 5-HT_{2C} receptor agonist is a 5-HT_{2A} receptor inverse agonist.

In some embodiments, the 5-HT_{2C} receptor agonist is a 5-HT_{2B} receptor inverse agonist.

In some embodiments, the 5-HT_{2C} receptor agonist is a 5-HT_{2A} receptor neutral antagonist.

In some embodiments, the 5-HT_{2C} receptor agonist is a 5-HT_{2B} receptor neutral antagonist.

In some embodiments, the 5-HT_{2C} receptor agonist is a 5-HT_{2A} receptor antagonist and a 5-HT_{2B} receptor antagonist.

In some embodiments, the 5-HT_{2C} receptor agonist is a 5-HT_{2A} receptor antagonist and a 5-HT_{2B} receptor partial agonist.

In some embodiments, the 5-HT_{2C} receptor agonist is a 5-HT_{2A} receptor antagonist and a 5-HT_{2B} receptor inverse agonist.

In some embodiments, the 5-HT_{2C} receptor agonist is a 5-HT_{2A} receptor antagonist and a 5-HT_{2B} receptor neutral antagonist.

In some embodiments, the 5-HT_{2C} receptor agonist is a 5-HT_{2A} receptor partial agonist and a 5-HT_{2B} receptor antagonist.

In some embodiments, the 5-HT_{2C} receptor agonist is a 5-HT_{2A} receptor partial agonist and a 5-HT_{2B} receptor partial agonist.

In some embodiments, the 5-HT_{2C} receptor agonist is a 5-HT_{2A} receptor partial agonist and a 5-HT_{2B} receptor inverse agonist.

In some embodiments, the 5-HT_{2C} receptor agonist is a 5-HT_{2A} receptor partial agonist and a 5-HT_{2B} receptor neutral antagonist.

In some embodiments, the 5-HT_{2C} receptor agonist is a 5-HT_{2A} receptor inverse agonist and a 5-HT_{2B} receptor antagonist.

In some embodiments, the 5-HT_{2C} receptor agonist is a 5-HT_{2A} receptor inverse agonist and a 5-HT_{2B} receptor partial agonist.

In some embodiments, the 5-HT_{2C} receptor agonist is a 5-HT_{2A} receptor inverse agonist and a 5-HT_{2B} receptor inverse agonist.

In some embodiments, the 5-HT_{2C} receptor agonist is a 5-HT_{2A} receptor inverse agonist and a 5-HT_{2B} receptor neutral antagonist.

In some embodiments, the 5-HT_{2C} receptor agonist is a 5-HT_{2A} receptor neutral antagonist and a 5-HT_{2B} receptor antagonist.

In some embodiments, the 5-HT_{2C} receptor agonist is a 5-HT_{2A} receptor neutral antagonist and a 5-HT_{2B} receptor partial agonist.

In some embodiments, the 5-HT_{2C} receptor agonist is a 5-HT_{2A} receptor neutral antagonist and a 5-HT_{2B} receptor inverse agonist.

In some embodiments, the 5-HT_{2C} receptor agonist is a 5-HT_{2A} receptor neutral antagonist and a 5-HT_{2B} receptor neutral antagonist.

In some embodiments, the 5-HT_{2C} receptor agonist is a 5-HT_{2A} receptor inverse agonist and a 5-HT_{2B} receptor inverse agonist.

In some embodiments, the 5-HT_{2C} receptor agonist is a 5-HT_{2A} receptor neutral antagonist and a 5-HT_{2B} receptor neutral antagonist.

In some embodiments, the 5-HT_{2C} receptor agonist is a 5-HT_{2A} receptor inverse agonist and a 5-HT_{2B} receptor antagonist.

In some embodiments, the 5-HT_{2C} receptor agonist is a 5-HT_{2A} receptor neutral antagonist and a 5-HT_{2B} receptor antagonist.

One aspect of the present invention pertains to 5-HT_{2C} receptor agonists for use by an individual or caregiver in: reducing a concentration of norepinephrine in said individual; or treating a disorder ameliorated by reducing a concentration of norepinephrine in said individual.

In some embodiments, the reducing or amelioration of the disorder occurs: within 56 days of administering first said dose; or within 7 days of administering first said dose.

In some embodiments, the reducing or amelioration of the disorder is not dependent upon concomitant weight-loss in said individual.

In some embodiments, the individual does not lose a substantial amount of weight during said reducing or amelioration of said disorder.

In some embodiments, the individual loses a substantial amount of weight during the reducing or amelioration of the disorder; and wherein the reducing or amelioration of the disorder is greater than the amount of reducing or amelioration expected by the individual or the caregiver solely as a result of the individual loosing the substantial amount of weight.

In some embodiments, the concentration is: a urine concentration; a blood concentration; a plasma concentration; a brain concentration; or a cerebrospinal fluid concentration.

In some embodiments, the reducing provides a reduced concentration of norepinephrine in the individual: at least about 10% lower than baseline; at least about 20% lower than baseline; at least about 30% lower than baseline; at least about 40% lower than baseline; or at least about 50% lower than baseline.

One aspect of the present invention pertains to 5-HT_{2C} receptor agonists of the present invention, for use by an individual or caregiver in reducing a concentration of norepinephrine in the individual and for maintaining the reduced concentration of norepinepherine.

In some embodiments, the reduced concentration is: a urine concentration; a blood concentration; a plasma concentration; a brain concentration; or a cerebrospinal fluid concentration.

In some embodiments, the disorder is selected from: hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

In some embodiments, the individual is a human.

In some embodiments, the 5-HT_{2C} receptor agonist of the present invention has an EC₅₀ of: less than about 10 μM at the 5-HT_{2C} receptor; less than about 1 μM at the 5-HT_{2C} receptor; less than about 100 nM at the 5-HT_{2C} receptor.

In some embodiments, the 5-HT_{2C} receptor agonist of the present invention is a selective 5-HT_{2C} receptor agonist.

In some embodiments, the ratio of the EC₅₀ of the 5-HT_{2C} receptor agonist at the 5-HT_{2C} receptor to the EC₅₀ of the 5-HT_{2C} receptor agonist at the 5-HT_{2A} receptor is: at least about 10:1; at least about 100:1; at least about 1000:1.

In some embodiments, the ratio of the EC₅₀ of the 5-HT_{2C} receptor agonist at the 5-HT_{2C} receptor to the EC₅₀ of the 5-HT_{2C} receptor agonist at the 5-HT_{2B} receptor is: at least about 10:1; at least about 100:1; at least about 1000:1.

In some embodiments, the 5-HT_{2C} receptor agonist is a partial agonist, an antagonist, an inverse agonist, or a neutral antagonist of the 5-HT_{2A} receptor.

In some embodiments, the 5-HT_{2C} receptor agonist is a partial agonist, an antagonist, an inverse agonist, or a neutral antagonist of the 5-HT_{2B} receptor.

In some embodiments, the 5-HT_{2C} receptor agonist is a small molecule.

In some embodiments, the 5-HT_{2C} receptor agonist is orally-bioavailable.

In some embodiments, the 5-HT_{2C} receptor agonist is selected from (R)-8-chloro-1-methyl-2,3,4,5-tetrahydro-1H-3-benzazepine, and pharmaceutically acceptable salts, solvates, and hydrates thereof.

In some embodiments, the 5-HT_{2C} receptor agonist is (R)-8-chloro-1-methyl-2,3,4,5-tetrahydro-1H-3-benzazepine hydrochloride salt hemihydrate.

### Methods

One aspect of the present invention pertains to methods for reducing a concentration of a catecholamine in an individual, comprising administering to the individual, by the individual or by a caregiver, one or more doses of a medicament comprising a therapeutically effective amount of a 5-HT_{2C} receptor agonist.

One aspect of the present invention pertains to methods for reducing a concentration of epinephrine in an individual, comprising administering to the individual, by the individual or by a caregiver, one or more doses of a medicament comprising a therapeutically effective amount of a 5-HT_{2C} receptor agonist.

One aspect of the present invention pertains to methods for reducing a concentration of norepinephrine in an individual, comprising administering to the individual, by the individual or by a caregiver, one or more doses of a medicament comprising a therapeutically effective amount of a 5-HT_{2C} receptor agonist.

In some embodiments, the method comprises administering to the individual, a single dose of the medicament.

In some embodiments, the method comprises short-term use of the medicament.

In some embodiments, the method comprises acute use of the medicament.

In some embodiments, the method comprises administering to the individual, a plurality of doses of the medicament.

In some embodiments, the method comprises administering to the individual, one or two doses of the medicament per day for at least one week.

In some embodiments, the method comprises administering to the individual, one or two doses of the medicament per day for at least one month.

In some embodiments, the reducing occurs within 56 days of administering the first dose.

In some embodiments, the reducing occurs within 7 days of administering the first dose.

In some embodiments, the reducing is not dependent upon concomitant weight-loss in the individual.

In some embodiments, the individual does not lose a substantial amount of weight during the reducing.

In some embodiments, the individual loses a substantial amount of weight during the reducing and the reducing is greater than the amount of reducing expected by the individual or said caregiver solely as a result of said individual loosing said substantial amount of weight.

In some embodiments, the concentration is a urine concentration.

In some embodiments, the concentration is a blood concentration.

In some embodiments, the concentration is a plasma concentration.

In some embodiments, the concentration is a brain concentration.

In some embodiments, the concentration is a cerebrospinal fluid concentration.

In some embodiments, the individual is hypernorepinephrinemic prior to the administering.

In some embodiments, the concentration of norepinephrine in the individual is at least 80 μg/24 h in urine prior to the administering.

In some embodiments, the concentration of norepinephrine in the individual is at least 600 pg/mL in blood prior to the administering.

In some embodiments, the reducing provides a reduced concentration of norepinephrine in the individual that is within the normal range.

In some embodiments, the reducing provides a reduced concentration of norepinephrine in the individual that is between about 15 and about 80 μg/24 h in urine.

In some embodiments, the reducing provides a reduced concentration of norepinephrine in the individual that is between about 0 and about 600 pg/mL in blood.

In some embodiments, the reducing provides a reduced concentration of norepinephrine in the individual at least about 10% lower than baseline.

In some embodiments, the reducing provides a reduced concentration of norepinephrine in the individual at least about 20% lower than baseline.

In some embodiments, the reducing provides a reduced concentration of norepinephrine in the individual at least about 30% lower than baseline.

In some embodiments, the reducing provides a reduced concentration of norepinephrine in the individual at least about 40% lower than baseline.

In some embodiments, the reducing provides a reduced concentration of norepinephrine in the individual at least about 50% lower than baseline.

One aspect of the present invention pertains to methods for maintaining a reduced concentration of a catecholamine in an individual, comprising administering to the individual, by the individual or by a caregiver, one or more doses of a medicament comprising a therapeutically effective amount of a 5-HT_{2C} receptor agonist.

One aspect of the present invention pertains to methods for maintaining a reduced concentration of epinephrine in an individual, comprising administering to the individual, by the individual or by a caregiver, one or more doses of a medicament comprising a therapeutically effective amount of a 5-HT_{2C} receptor agonist.

One aspect of the present invention pertains to methods for maintaining a reduced concentration of norepinepherine in an individual, comprising administering to the individual, by the individual or by a caregiver, one or more doses of a medicament comprising a therapeutically effective amount of a 5-HT_{2C} receptor agonist.

In some embodiments, the reduced concentration is a urine concentration.

In some embodiments, the reduced concentration is a blood concentration.

In some embodiments, the reduced concentration is a plasma concentration.

In some embodiments, the reduced concentration is a brain concentration.

In some embodiments, the reduced concentration is a cerebrospinal fluid concentration.

One aspect of the present invention pertains to methods for treating a disorder ameliorated by reduction of a concentration of a catecholamine in an individual, comprising administering to the individual, by the individual or by a caregiver, one or more doses of a medicament comprising a therapeutically effective amount of a 5-HT_{2C} receptor agonist.

One aspect of the present invention pertains to methods for treating a disorder ameliorated by reduction of a concentration of epinephrine in an individual, comprising administering to the individual, by the individual or by a caregiver, one or more doses of a medicament comprising a therapeutically effective amount of a 5-HT_{2C} receptor agonist.

One aspect of the present invention pertains to methods for treating a disorder ameliorated by reduction of a concentration of norepinephrine in an individual, comprising administering to the individual, by the individual or by a caregiver, one or more doses of a medicament comprising a therapeutically effective amount of a 5-HT_{2C} receptor agonist.

In some embodiments, the method comprises administering to the individual, a plurality of doses of the medicament.

In some embodiments, the method comprises administering to the individual, a single dose of the medicament.

In some embodiments, the method comprises short-term use of the medicament.

In some embodiments, the method comprises acute use of the medicament.

In some embodiments, the method comprises administering to the individual, one or two doses of the medicament per day for at least one week.

In some embodiments, the method comprises administering to the individual, one or two doses of the medicament per day for at least one month.

In some embodiments, amelioration of the disorder occurs within 56 days of administering the first dose.

In some embodiments, amelioration of the disorder occurs within 7 days of administering the first dose.

In some embodiments, the individual does not lose a substantial amount of weight during the reducing.

In some embodiments, the individual loses a substantial amount of weight during the reducing and the disorder is ameliorated more than would be expected by the individual or the caregiver, solely as a result of the individual loosing the substantial amount of weight.

In some embodiments, the disorder is selected from: hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

In some embodiments, the disorder is elevated heart rate.

In some embodiments, the disorder is vasoconstriction.

In some embodiments, the disorder is acute pulmonary vasoconstriction.

In some embodiments, the disorder is hypertension.

In some embodiments, the disorder is heart failure.

In some embodiments, the disorder is cardiac dysfunction after stroke.

In some embodiments, the disorder is cardiac arrhythmia.

In some embodiments, the disorder is metabolic syndrome.

In some embodiments, the disorder is abnormal lipid metabolism.

In some embodiments, the disorder is hyperthermia.

In some embodiments, the disorder is Cushing syndrome.

In some embodiments, the disorder is pheochromocytoma.

In some embodiments, the disorder is epilepsy.

In some embodiments, the disorder is obstructive sleep apnea.

In some embodiments, the disorder is insomnia.

In some embodiments, the disorder is glaucoma.

In some embodiments, the disorder is osteoarthritis.

In some embodiments, the disorder is rheumatoid arthritis.

In some embodiments, the disorder is asthma.

In some embodiments, the individual is a human.

In any of the methods of the present invention, there is the optional step of determining a concentration of norepinephrine in an individual before administering to the individual the therapeutically effective amount of the 5-HT_{2C} receptor agonist.

In any of the methods of the present invention, there is the optional step of diagnosing hypernorepinephrinemia in an individual before administering to the individual the therapeutically effective amount of the 5-HT_{2C} receptor agonist.

It is understood by one of skill in the art that a concentration of norepinepherine in an individual can optionally be measured directly, such as, by monitoring the individual's blood norepinepherine or urine norepinepherine.

It is understood by one of skill in the art that a concentration of norepinepherine in an individual can optionally be measured indirectly, such as, by monitoring a surrogate for the individual's norepinepherine concentration, such as the individual's blood pressure.

In some embodiments, the 5-HT_{2C} receptor agonist has an EC₅₀ of less than about 10 μM at the 5-HT_{2C} receptor.

In some embodiments, the 5-HT_{2C} receptor agonist has an EC₅₀ of less than about 1 μM at the 5-HT_{2C} receptor.

In some embodiments, the 5-HT_{2C} receptor agonist has an EC₅₀ of less than about 100 nM at the 5-HT_{2C} receptor.

In some embodiments, the 5-HT_{2C} receptor is a human 5-HT_{2C} receptor.

In some embodiments, the 5-HT_{2C} receptor agonist is a selective 5-HT_{2C} receptor agonist.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2A} receptor is at least about 1000:1.

In some embodiments, the 5-HT_{2A} receptor is a human 5-HT_{2A} receptor; and the 5-HT_{2C} receptor is a human 5-HT_{2C} receptor.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 10:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 100:1.

In some embodiments, the ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is at least about 1000:1.

In some embodiments, the 5-HT_{2B} receptor is a human 5-HT_{2B} receptor; and the 5-HT_{2C} receptor is a human 5-HT_{2C} receptor.

In some embodiments, the selective 5-HT_{2C} receptor agonist is a partial agonist, an antagonist, an inverse agonist, or a neutral antagonist of the 5-HT_{2A} receptor.

In some embodiments, the selective 5-HT_{2C} receptor agonist is a partial agonist, an antagonist, an inverse agonist, or a neutral antagonist of the 5-HT_{2B} receptor.

In some embodiments, the selective 5-HT_{2C} receptor agonist is a partial agonist, an antagonist, an inverse agonist, or a neutral antagonist of the 5-HT_{2A} receptor; and a partial agonist, an antagonist, an inverse agonist, or a neutral antagonist of the 5-HT_{2B} receptor.

In some embodiments, the 5-HT_{2A} receptor is a human 5-HT_{2A} receptor; the 5-HT_{2B} receptor is a human 5-HT_{2B} receptor; and said 5-HT_{2C} receptor is a human 5-HT_{2C} receptor.

In some embodiments, the 5-HT_{2C} receptor agonist is a small molecule.

In some embodiments, the 5-HT_{2C} receptor agonist is orally bioavailable.

One aspect of the present invention pertains to methods for: reducing a concentration of norepinephrine in an individual; or treating a disorder ameliorated by reducing a concentration of norepinephrine in an individual; comprising administering to the individual, by the individual or by a caregiver, one or more doses of a medicament comprising a therapeutically effective amount of a 5-HT_{2C} receptor agonist.

In some embodiments, the method comprises administering to the individual a plurality of doses of the medicament.

In some embodiments, the method comprises administering to the individual one or two doses of the medicament per day for: at least one week; or at least one month.

In some embodiments, the reducing, or amelioration of the disorder occurs: within 56 days of administering first the dose; or within 7 days of administering first the dose.

In some embodiments, the reducing, or amelioration of the disorder is not dependent upon concomitant weight-loss in the individual.

In some embodiments, the individual does not lose a substantial amount of weight during the reducing or amelioration of the disorder.

In some embodiments, the individual loses a substantial amount of weight during the reducing or amelioration of the disorder; and wherein the reducing or amelioration of the disorder is greater than the amount of reducing or amelioration expected by the individual or the caregiver solely as a result of the individual loosing the substantial amount of weight.

In some embodiments, the concentration is: a urine concentration; a blood concentration; a plasma concentration; a brain concentration; or a cerebrospinal fluid concentration.

In some embodiments, the reducing provides a reduced concentration of norepinephrine in the individual: at least about 10% lower than baseline; at least about 20% lower than baseline; at least about 30% lower than baseline; at least about 40% lower than baseline; or at least about 50% lower than baseline.

One aspect of the present invention pertains to methods for maintaining a reduced concentration of norepinepherine, comprising administering to an individual, by the individual or by a caregiver, one or more doses of a medicament comprising a therapeutically effective amount of a 5-HT_{2C} receptor agonist.

In some embodiments, the reduced concentration is: a urine concentration; a blood concentration; a plasma concentration; a brain concentration; or a cerebrospinal fluid concentration.

In some embodiments, individual is a human.

In some embodiments, 5-HT_{2C} receptor agonist has an EC₅₀ of: less than about 10 μM at the 5-HT_{2C} receptor; less than about 1 μM at the 5-HT_{2C} receptor; or less than about 100 nM at the 5-HT_{2C} receptor.

In some embodiments, 5-HT_{2C} receptor agonist is a selective 5-HT_{2C} receptor agonist.

In some embodiments, EC₅₀ of the selective 5-HT_{2C} receptor agonist at the 5-HT_{2C} receptor to the EC₅₀ of the selective 5-HT_{2C} receptor agonist at the 5-HT_{2A} receptor is: at least about 10:1; at least about 100:1; or at least about 1000:1.

In some embodiments, ratio of the EC₅₀ of the selective 5-HT_{2C} receptor agonist at the 5-HT_{2C} receptor to the EC₅₀ of the selective 5-HT_{2C} receptor agonist at the 5-HT_{2B} receptor is: at least about 10:1; at least about 100:1; or at least about 1000:1.

In some embodiments, selective 5-HT_{2C} receptor agonist is a partial agonist, an antagonist, an inverse agonist, or a neutral antagonist of the 5-HT_{2A} receptor.

In some embodiments, selective 5-HT_{2C} receptor agonist is a partial agonist, an antagonist, an inverse agonist, or a neutral antagonist of the 5-HT_{2B} receptor.

In some embodiments, the 5-HT_{2C} receptor agonist is a small molecule.

In some embodiments, the 5-HT_{2C} receptor agonist is orally-bioavailable.

In some embodiments, the selective 5-HT_{2C} receptor agonist is selected from 8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is (R)-8-chloro-1-methyl-2,3,4,5-tetrahydro-1H-3-benzazepine hydrochloride salt hemihydrate.

In some embodiments, the selective 5-HT_{2C} receptor agonist is selected from 8-chloro-1-methyl-2,3,4,5-tetrahydro-1H-3-benzazepine, and pharmaceutically acceptable salts, solvates, and hydrates thereof.

In some embodiments, the 5-HT_{2C} receptor agonist is selected from (R)-8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is selected from (R)-8-chloro-1-methyl-2,3,4,5-tetrahydro-1H-3-benzazepine hydrochloride salt, and pharmaceutically acceptable solvates and hydrates thereof.

In some embodiments, the selective 5-HT_{2C} receptor agonist is (R)-8-chloro-1-methyl-2,3,4,5-tetrahydro-1H-3-benzazepine hydrochloride salt hemihydrate.

In some embodiments, the terms “(R)-8-chloro-1-methyl-2,3,4,5-tetrahydro-1H-3-benzazepine or a pharmaceutically acceptable salt, solvate, or hydrate thereof” and “(R)-8-chloro-1-methyl-2,3,4,5-tetrahydro-1H-3-benzazepine, and pharmaceutically acceptable salts, solvates, and hydrates thereof” as used herein encompass any one of the following salts, or a Markush group comprising any combination of the following salts:
(R)-8-chloro-1-methyl-2,3,4,5-tetrahydro-1H-3-benzazepine hydroiodide salt;
(R)-8-chloro-1-methyl-2,3,4,5-tetrahydro-1H-3-benzazepine maleate salt;
(R)-8-chloro-1-methyl-2,3,4,5-tetrahydro-1H-3-benzazepine fumarate salt; and
(R)-8-chloro-1-methyl-2,3,4,5-tetrahydro-1H-3-benzazepine hemifumarate salt;
(R)-8-chloro-1-methyl-2,3,4,5-tetrahydro-1H-3-benzazepine orotate salt;
(R)-8-chloro-1-methyl-2,3,4,5-tetrahydro-1H-3-benzazepine di-acetamidobenzoate salt-cocrystal;
(R)-8-chloro-1-methyl-2,3,4,5-tetrahydro-1H-3-benzazepine trans-cinnamate salt;
(R)-8-chloro-1-methyl-2,3,4,5-tetrahydro-1H-3-benzazepine heminapadisilate salt;
(R)-8-chloro-1-methyl-2,3,4,5-tetrahydro-1H-3-benzazepine (±)-mandelate salt;
(R)-8-chloro-1-methyl-2,3,4,5-tetrahydro-1H-3-benzazepine hemipamoate salt;
(R)-8-chloro-1-methyl-2,3,4,5-tetrahydro-1H-3-benzazepine (1S)-(+)-10-camsylate salt;
(R)-8-chloro-1-methyl-2,3,4,5-tetrahydro-1H-3-benzazepine hemi-L-malate salt;
(R)-8-chloro-1-methyl-2,3,4,5-tetrahydro-1H-3-benzazepine L-glutamate salt;
(R)-8-chloro-1-methyl-2,3,4,5-tetrahydro-1H-3-benzazepine L-aspartate salt;
(R)-8-chloro-1-methyl-2,3,4,5-tetrahydro-1H-3-benzazepine hemimucate salt;
(R)-8-chloro-1-methyl-2,3,4,5-tetrahydro-1H-3-benzazepine pyroglutamate salt;
(R)-8-chloro-1-methyl-2,3,4,5-tetrahydro-1H-3-benzazepine glucuronate salt; and
(R)-8-chloro-1-methyl-2,3,4,5-tetrahydro-1H-3-benzazepine di-camphorate salt;
(R)-8-chloro-1-methyl-2,3,4,5-tetrahydro-1H-3-benzazepine bisulfate salt;
(R)-8-chloro-1-methyl-2,3,4,5-tetrahydro-1H-3-benzazepine hemisulfate salt;
(R)-8-chloro-1-methyl-2,3,4,5-tetrahydro-1H-3-benzazepine mesylate salt;
(R)-8-chloro-1-methyl-2,3,4,5-tetrahydro-1H-3-benzazepine hydrobromide salt;
(R)-8-chloro-1-methyl-2,3,4,5-tetrahydro-1H-3-benzazepine nitrate salt;
(R)-8-chloro-1-methyl-2,3,4,5-tetrahydro-1H-3-benzazepine sesqui-oxalate salt-cocrystal;
(R)-8-chloro-1-methyl-2,3,4,5-tetrahydro-1H-3-benzazepine adipate salt;
(R)-8-chloro-1-methyl-2,3,4,5-tetrahydro-1H-3-benzazepine malonate salt;
(R)-8-chloro-1-methyl-2,3,4,5-tetrahydro-1H-3-benzazepine hemimalonate salt;
(R)-8-chloro-1-methyl-2,3,4,5-tetrahydro-1H-3-benzazepine glycolate salt;
(R)-8-chloro-1-methyl-2,3,4,5-tetrahydro-1H-3-benzazepine hemi-edisylate salt;
(R)-8-chloro-1-methyl-2,3,4,5-tetrahydro-1H-3-benzazepine phosphate salt;
(R)-8-chloro-1-methyl-2,3,4,5-tetrahydro-1H-3-benzazepine citrate salt;
(R)-8-chloro-1-methyl-2,3,4,5-tetrahydro-1H-3-benzazepine hemi-oxalate salt;
(R)-8-chloro-1-methyl-2,3,4,5-tetrahydro-1H-3-benzazepine succinate salt; and
(R)-8-chloro-1-methyl-2,3,4,5-tetrahydro-1H-3-benzazepine oxoglutarate salt; and pharmaceutically acceptable solvates and hydrates thereof.

In some embodiments, the terms “(R)-8-chloro-1-methyl-2,3,4,5-tetrahydro-1H-3-benzazepine or a pharmaceutically acceptable salt, solvate, or hydrate thereof” and “(R)-8-chloro-1-methyl-2,3,4,5-tetrahydro-1H-3-benzazepine, and pharmaceutically acceptable salts, solvates, and hydrates thereof” as used herein encompass any one of the following salts, or a Markush group comprising any combination of the following salts:
(R)-8-chloro-1-methyl-2,3,4,5-tetrahydro-1H-3-benzazepine hydroiodide salt;
(R)-8-chloro-1-methyl-2,3,4,5-tetrahydro-1H-3-benzazepine maleate salt;
(R)-8-chloro-1-methyl-2,3,4,5-tetrahydro-1H-3-benzazepine fumarate salt;
(R)-8-chloro-1-methyl-2,3,4,5-tetrahydro-1H-3-benzazepine hemifumarate salt;
(R)-8-chloro-1-methyl-2,3,4,5-tetrahydro-1H-3-benzazepine orotate salt;
(R)-8-chloro-1-methyl-2,3,4,5-tetrahydro-1H-3-benzazepine orotate salt hydrate;
(R)-8-chloro-1-methyl-2,3,4,5-tetrahydro-1H-3-benzazepine di-4-acetamidobenzoate salt-cocrystal methyl ethyl ketone solvate;
(R)-8-chloro-1-methyl-2,3,4,5-tetrahydro-1H-3-benzazepine trans-cinnamate salt;
(R)-8-chloro-1-methyl-2,3,4,5-tetrahydro-1H-3-benzazepine heminapadisilate salt;
(R)-8-chloro-1-methyl-2,3,4,5-tetrahydro-1H-3-benzazepine heminapadisilate salt solvate 1;
(R)-8-chloro-1-methyl-2,3,4,5-tetrahydro-1H-3-benzazepine heminapadisilate salt solvate 2;
(R)-8-chloro-1-methyl-2,3,4,5-tetrahydro-1H-3-benzazepine (±)-mandelate salt hydrate;
(R)-8-chloro-1-methyl-2,3,4,5-tetrahydro-1H-3-benzazepine hemipamoate salt hydrate;
(R)-8-chloro-1-methyl-2,3,4,5-tetrahydro-1H-3-benzazepine (1S)-(+)-10-camsylate salt;
(R)-8-chloro-1-methyl-2,3,4,5-tetrahydro-1H-3-benzazepine hemi-L-malate salt;
(R)-8-chloro-1-methyl-2,3,4,5-tetrahydro-1H-3-benzazepine L-glutamate salt;
(R)-8-chloro-1-methyl-2,3,4,5-tetrahydro-1H-3-benzazepine L-aspartate salt;
(R)-8-chloro-1-methyl-2,3,4,5-tetrahydro-1H-3-benzazepine hemimucate salt;
(R)-8-chloro-1-methyl-2,3,4,5-tetrahydro-1H-3-benzazepine pyroglutamate salt;
(R)-8-chloro-1-methyl-2,3,4,5-tetrahydro-1H-3-benzazepine glucuronate salt;
(R)-8-chloro-1-methyl-2,3,4,5-tetrahydro-1H-3-benzazepine di-camphorate salt solvate;
(R)-8-chloro-1-methyl-2,3,4,5-tetrahydro-1H-3-benzazepine bisulfate salt;
(R)-8-chloro-1-methyl-2,3,4,5-tetrahydro-1H-3-benzazepine hemisulfate salt hydrate;
(R)-8-chloro-1-methyl-2,3,4,5-tetrahydro-1H-3-benzazepine mesylate salt;
(R)-8-chloro-1-methyl-2,3,4,5-tetrahydro-1H-3-benzazepine hydrobromide salt hemihydrate;
(R)-8-chloro-1-methyl-2,3,4,5-tetrahydro-1H-3-benzazepine nitrate salt;
(R)-8-chloro-1-methyl-2,3,4,5-tetrahydro-1H-3-benzazepine sesqui-oxalate salt-cocrystal;
(R)-8-chloro-1-methyl-2,3,4,5-tetrahydro-1H-3-benzazepine adipate salt;
(R)-8-chloro-1-methyl-2,3,4,5-tetrahydro-1H-3-benzazepine malonate salt;
(R)-8-chloro-1-methyl-2,3,4,5-tetrahydro-1H-3-benzazepine hemimalonate salt;
(R)-8-chloro-1-methyl-2,3,4,5-tetrahydro-1H-3-benzazepine glycolate salt;
(R)-8-chloro-1-methyl-2,3,4,5-tetrahydro-1H-3-benzazepine hemi-edisylate salt;
(R)-8-chloro-1-methyl-2,3,4,5-tetrahydro-1H-3-benzazepine phosphate salt;
(R)-8-chloro-1-methyl-2,3,4,5-tetrahydro-1H-3-benzazepine citrate salt hemihydrate;
(R)-8-chloro-1-methyl-2,3,4,5-tetrahydro-1H-3-benzazepine hemi-oxalate salt;
(R)-8-chloro-1-methyl-2,3,4,5-tetrahydro-1H-3-benzazepine succinate salt;
(R)-8-chloro-1-methyl-2,3,4,5-tetrahydro-1H-3-benzazepine oxoglutarate salt; and
(R)-8-chloro-1-methyl-2,3,4,5-tetrahydro-1H-3-benzazepine oxoglutarate salt solvate.

The preceding salts were prepared and characterized using the following experimental procedures and physicochemical data.

(R)-8-Chloro-1-methyl-2,3,4,5-tetrahydro-1H-3-benzazepine hydroiodide salt was prepared by the dropwise addition of one equivalent of aqueous HI (˜57%) to a solution of (R)-8-chloro-1-methyl-2,3,4,5-tetrahydro-1H-3-benzazepine free base in isopropyl acetate. A precipitate formed after 7 days stirring with evaporation. The solid was slurried in ethyl acetate with ˜3% water added for 5 h. The solid was recovered by centrifuge filtration (10,000 rpm for 1 minute, nylon filter). (R)-8-Chloro-1-methyl-2,3,4,5-tetrahydro-1H-3-benzazepine hydroiodide salt had an extrapolated melting onset temperature by DSC of 155-156° C.

(R)-8-Chloro-1-methyl-2,3,4,5-tetrahydro-1H-3-benzazepine maleate salt was prepared by dropwise addition of a solution of 1 or 2 equivalents of maleic acid in methanol to a solution of (R)-8-chloro-1-methyl-2,3,4,5-tetrahydro-1H-3-benzazepine free base in isopropyl acetate or acetonitrile with vigorous stirring. The resulting slurry was heated to 60° C. and held at that temperature for ˜1 h before it was cooled to room temperature and stirred overnight. The title salt was recovered by filtration, washed with isopropyl acetate or acetonitrile and dried on the filter before characterization. (R)-8-Chloro-1-methyl-2,3,4,5-tetrahydro-1H-3-benzazepine maleate salt had an extrapolated melting onset temperature by DSC of about 166° C.

(R)-8-Chloro-1-methyl-2,3,4,5-tetrahydro-1H-3-benzazepine fumarate salt was prepared by dropwise addition of an equimolar amount of fumaric acid in 1:1 water:EtOH (˜0.6 M) to a solution of (R)-8-chloro-1-methyl-2,3,4,5-tetrahydro-1H-3-benzazepine in isopropyl acetate with vigorous stirring. The resulting suspension was heated to 60° C., held at that temperature for 1 h, and then allowed to cool to ambient temperature while stirring overnight. The mixture was filtered and the solid was washed with isopropyl acetate and dried on the filter. Alternatively, (R)-8-chloro-1-methyl-2,3,4,5-tetrahydro-1H-3-benzazepine fumarate salt was prepared by adding either a half molar or an equimolar amount of dry solid fumaric acid to solution of (R)-8-chloro-1-methyl-2,3,4,5-tetrahydro-1H-3-benzazepine in isopropyl acetate. The mixture was slurried at ˜60° C. and stirred for ˜2 h. The heat source was removed and the mixture was left to stir for 3 days at ˜26° C. The solid precipitate was recovered by filtration, and then re-slurried for ˜24 h in water or ethanol. The solid was recovered by filtration and slurried for an additional 4 days in n-propanol, acetonitrile, or water. (R)-8-Chloro-1-methyl-2,3,4,5-tetrahydro-1H-3-benzazepine fumarate salt had an extrapolated melting onset temperature by DSC of 218-219° C.

(R)-8-Chloro-1-methyl-2,3,4,5-tetrahydro-1H-3-benzazepine hemifumarate salt was prepared by dropwise addition of a half-molar amount of fumaric acid in 1:1 water:EtOH (˜0.6 M) to a solution of (R)-8-chloro-1-methyl-2,3,4,5-tetrahydro-1H-3-benzazepine in isopropyl acetate with vigorous stirring. A suspension resulted. It was heated to 60° C., held at that temperature for 1 h, and then the heat source was removed and the sample was allowed to cool to ambient temperature while stirring overnight. The suspension was filtered and the solid was washed with isopropyl acetate and dried on the filter. (R)-8-Chloro-1-methyl-2,3,4,5-tetrahydro-1H-3-benzazepine hemifumarate salt had an extrapolated melting onset temperature by DSC of 158° C.

(R)-8-Chloro-1-methyl-2,3,4,5-tetrahydro-1H-3-benzazepine orotate salt was prepared by addition of one equivalent of orotic acid to a solution of (R)-8-chloro-1-methyl-2,3,4,5-tetrahydro-1H-3-benzazepine in isopropanol, ethyl acetate, or acetone at 60° C. Orotic acid, at 60° C., was added drop-wise, in the corresponding solvent, with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine orotate salt had an extrapolated initial melting onset temperature by DSC of 236° C.

(R)-8-Chloro-1-methyl-2,3,4,5-tetrahydro-1H-3-benzazepine orotate salt hydrate was prepared by addition of one equivalent of orotic acid to a solution of (R)-8-chloro-1-methyl-2,3,4,5-tetrahydro-1H-3-benzazepine in acetonitrile or isopropanol at 60° C. Orotic acid, at 60° C., was added drop-wise, in the corresponding solvent, with vigorous stirring. Precipitation occurred immediately and the suspension was allowed to cool and stir overnight. Compound 1 orotate salt hydrate prepared in isopropanol consisted of a mixture of the anhydrous and hydrated forms which was converted to the hydrated form by slurring in isopropanol for two days. Alternatively, (R)-8-Chloro-1-methyl-2,3,4,5-tetrahydro-1H-3-benzazepine orotate salt hydrate was prepared by slurrying anhydrous (R)-8-chloro-1-methyl-2,3,4,5-tetrahydro-1H-3-benzazepine orotate salt in water. (R)-8-Chloro-1-methyl-2,3,4,5-tetrahydro-1H-3-benzazepine orotate salt hydrate had an extrapolated melt/recrystallization onset temperature by DSC of 173° C.

(R)-8-Chloro-1-methyl-2,3,4,5-tetrahydro-1H-3-benzazepine di-4-acetamidobenzoate salt-cocrystal methyl ethyl ketone solvate was prepared by combining one equivalent of 4-acetamidobenzoic acid with (R)-8-chloro-1-methyl-2,3,4,5-tetrahydro-1H-3-benzazepine in n-propanol or methanol at 50° C. then cooling slowly and stirring overnight. The resulting clear solution was evaporated to a mixture of oil and solids. Upon trituration with MEK a white solid formed and was filtered and dried. (R)-8-Chloro-1-methyl-2,3,4,5-tetrahydro-1H-3-benzazepine di-4-acetamidobenzoate salt-cocrystal methyl ethyl ketone solvate had an extrapolated melting/desolvation onset temperature by DSC of 113° C.

(R)-8-Chloro-1-methyl-2,3,4,5-tetrahydro-1H-3-benzazepine trans-cinnamate salt was prepared by combining one equivalent of trans-cinnamic acid with (R)-8-chloro-1-methyl-2,3,4,5-tetrahydro-1H-3-benzazepine in acetonitrile at 50° C. The sample was cooled slowly and stirred overnight. The resulting white solid was isolated by filtration and dried. Similar samples prepared in isopropanol, acetone or THF produced white solids only after removal of solvent and trituration with MTBE. (R)-8-Chloro-1-methyl-2,3,4,5-tetrahydro-1H-3-benzazepine trans-cinnamate salt had an extrapolated melting onset temperature by DSC of 106° C.

(R)-8-Chloro-1-methyl-2,3,4,5-tetrahydro-1H-3-benzazepine heminapadisilate salt was prepared by addition of a molar equivalent of naphthalene-1,5-disulfonic acid to a solution of (R)-8-chloro-1-methyl-2,3,4,5-tetrahydro-1H-3-benzazepine in isopropanol or acetonitrile at 60° C. Naphthalene-1,5-disulfonic acid, at 60° C., was added drop-wise, in the corresponding solvent, with vigorous stirring. Precipitation occurred immediately in acetonitrile and the suspension was allowed to cool and stir overnight. Addition of water precipitated the salt in isopropanol and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine heminapadisilate salt had an extrapolated melting onset temperature by DSC of about 266° C.

(R)-8-Chloro-1-methyl-2,3,4,5-tetrahydro-1H-3-benzazepine heminapadisilate salt solvate 1 was prepared by addition of one equivalent of naphthalene-1,5-disulfonic acid to a solution of (R)-8-chloro-1-methyl-2,3,4,5-tetrahydro-1H-3-benzazepine in ethyl acetate at 60° C. Naphthalene-1,5-disulfonic acid in ethyl acetate, at 60° C., was added dropwise with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine heminapadisilate salt solvate 1 had an extrapolated desolvation onset temperature by DSC of about 101° C.

(R)-8-Chloro-1-methyl-2,3,4,5-tetrahydro-1H-3-benzazepine heminapadisilate salt solvate 2 was prepared by the addition of one equivalent of naphthalene-1,5-disulfonic acid to a solution of (R)-8-chloro-1-methyl-2,3,4,5-tetrahydro-1H-3-benzazepine in acetone at 60° C. Naphthalene-1,5-disulfonic acid in acetone at 60° C. was added dropwise with vigorous stirring. A yellow oil precipitated and the suspension was allowed to cool and stir overnight. A white precipitate was observed after stirring overnight. The resulting solid was recovered by filtration and air-dried in a fume hood overnight. (R)-8-Chloro-1-methyl-2,3,4,5-tetrahydro-1H-3-benzazepine heminapadisilate salt solvate 2 had an extrapolated desolvation onset temperature by DSC of about 129° C.

(R)-8-Chloro-1-methyl-2,3,4,5-tetrahydro-1H-3-benzazepine (±)-mandelate salt hydrate was prepared by the addition of one equivalent of (±)-mandelic acid to a solution of (R)-8-chloro-1-methyl-2,3,4,5-tetrahydro-1H-3-benzazepine in acetonitrile, ethyl acetate, or acetone at 60° C. (±)-Mandelic acid, at 60° C., was added dropwise, in the corresponding solvent, with vigorous stirring. Addition of water to these three samples precipitated the salt and it was allowed to cool and stir overnight. The resulting solids were recovered by filtration and air-dried in a fume hood overnight. (R)-8-Chloro-1-methyl-2,3,4,5-tetrahydro-1H-3-benzazepine (±)-mandelate salt hydrate had an extrapolated desolvation onset temperature by DSC of about 74° C.

(R)-8-Chloro-1-methyl-2,3,4,5-tetrahydro-1H-3-benzazepine hemipamoate salt hydrate was prepared by the addition of 0.25 molar equivalents of pamoic acid to a solution of (R)-8-chloro-1-methyl-2,3,4,5-tetrahydro-1H-3-benzazepine in isopropanol, acetonitrile, ethyl acetate, or acetone at 60° C. Pamoic acid, at 60° C., was added dropwise, in the corresponding solvent, with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine hemipamoate salt hydrate had an extrapolated melting onset temperature by DSC of about 244° C.

(R)-8-Chloro-1-methyl-2,3,4,5-tetrahydro-1H-3-benzazepine (1S)-(+)-10-camsylate salt was prepared by the dropwise addition of 1 mole equivalent of ˜3.6 M aqueous (1S)-(+)-10-camphorsulfonic acid to a solution of (R)-8-chloro-1-methyl-2,3,4,5-tetrahydro-1H-3-benzazepine in acetonitrile with vigorous stirring. Immediate precipitation was observed and the solid was collected by filtration and washed with isopropyl alcohol. (R)-8-Chloro-1-methyl-2,3,4,5-tetrahydro-1H-3-benzazepine (1S)-(+)-10-camsylate salt had an extrapolated melting onset temperature by DSC of about 176° C.

(R)-8-Chloro-1-methyl-2,3,4,5-tetrahydro-1H-3-benzazepine hemi-L-malate salt was prepared by the dropwise addition of L-malic acid (0.5 eq.), either in solution in hot MeOH or as a solid, to a solution of (R)-8-chloro-1-methyl-2,3,4,5-tetrahydro-1H-3-benzazepine in isopropyl acetate. The mixture was heated to ˜60° C. and held at that temperature for ˜1 h. The mixture was then allowed to cool to room temperature and stirred for 1-3 days. The solid product was isolated by vacuum filtration and dried on the filter or in an oven at 40° C. (R)-8-Chloro-1-methyl-2,3,4,5-tetrahydro-1H-3-benzazepine hemi-L-malate salt had an extrapolated melting onset temperature by DSC of 155-156° C.

(R)-8-Chloro-1-methyl-2,3,4,5-tetrahydro-1H-3-benzazepine L-glutamate salt was prepared by addition of L-glutamic acid (0.5-1 eq.) in hot EtOH/H₂O (˜2:1) to a solution of (R)-8-chloro-1-methyl-2,3,4,5-tetrahydro-1H-3-benzazepine in isopropyl acetate, followed by evaporation of the solvent overnight to produce a solid. The solid was slurried in isopropyl acetate and then isolated by filtration. Alternatively, (R)-8-Chloro-1-methyl-2,3,4,5-tetrahydro-1H-3-benzazepine L-glutamate salt was prepared by addition of a solution of L-glutamic acid (1 eq.) in hot H₂O to a solution of (R)-8-chloro-1-methyl-2,3,4,5-tetrahydro-1H-3-benzazepine. The product crystallized without the need for evaporation of the solvent. (R)-8-Chloro-1-methyl-2,3,4,5-tetrahydro-1H-3-benzazepine L-glutamate salt had an extrapolated melting onset temperature by DSC of about 187° C.

(R)-8-Chloro-1-methyl-2,3,4,5-tetrahydro-1H-3-benzazepine L-aspartate salt was prepared by addition of a solution of (R)-8-chloro-1-methyl-2,3,4,5-tetrahydro-1H-3-benzazepine in either acetone or acetonitrile to one equivalent of aspartic acid solid. The mixture was heated to 50° C. then slow-cooled and stirred overnight. (R)-8-Chloro-1-methyl-2,3,4,5-tetrahydro-1H-3-benzazepine L-aspartate salt had an extrapolated melting onset temperature by DSC of about 174° C.

(R)-8-Chloro-1-methyl-2,3,4,5-tetrahydro-1H-3-benzazepine hemimucate salt was synthesized from (R)-8-chloro-1-methyl-2,3,4,5-tetrahydro-1H-3-benzazepine (2 equivalents) and mucic acid (1 equivalent) in THF, acetone or IPA (−10 mg/mL) with 4% water. (R)-8-Chloro-1-methyl-2,3,4,5-tetrahydro-1H-3-benzazepine hemimucate salt had an extrapolated melting onset temperature by DSC of about 208° C.

(R)-8-Chloro-1-methyl-2,3,4,5-tetrahydro-1H-3-benzazepine glucuronate salt was prepared by addition of a molar equivalent of D-glucuronic acid to a solution of (R)-8-chloro-1-methyl-2,3,4,5-tetrahydro-1H-3-benzazepine in isopropanol, acetonitrile, ethyl acetate, or acetone at 60° C. D-glucuronic acid, dissolved in the corresponding solvent at 60° C., was added dropwise with vigorous stirring. Precipitation occurred immediately and the suspension was allowed to cool and stir overnight. The resulting solid was recovered by filtration and dried in a fume hood overnight. (R)-8-Chloro-1-methyl-2,3,4,5-tetrahydro-1H-3-benzazepine glucuronate salt had an extrapolated melting onset temperature by DSC of about 164° C.

(R)-8-Chloro-1-methyl-2,3,4,5-tetrahydro-1H-3-benzazepine pyroglutamate salt was prepared by combining one equivalent of pyroglutamic acid with (R)-8-chloro-1-methyl-2,3,4,5-tetrahydro-1H-3-benzazepine in ethyl acetate at 60° C. then cooling slowly and stirring overnight. The resulting white solid was isolated by filtration and dried. (R)-8-Chloro-1-methyl-2,3,4,5-tetrahydro-1H-3-benzazepine pyroglutamate salt had an extrapolated melting onset temperature by DSC of about 139° C.

(R)-8-Chloro-1-methyl-2,3,4,5-tetrahydro-1H-3-benzazepine di-camphorate salt solvate was prepared by combining equal molar amounts of (R)-8-chloro-1-methyl-2,3,4,5-tetrahydro-1H-3-benzazepine and (1R,3S)-(+)-camphoric acid in ethyl acetate with 4% water. The solution was heated to 50° C. then slowly cooled. Upon cooling the sample was a clear solution and did not change after addition of MTBE. The sample was evaporated to a clear oil which formed a white solid after standing at room temperature. (R)-8-Chloro-1-methyl-2,3,4,5-tetrahydro-1H-3-benzazepine di-camphorate salt had an extrapolated melting onset temperature by DSC of about 90° C.

(R)-8-Chloro-1-methyl-2,3,4,5-tetrahydro-1H-3-benzazepine bisulfate salt was prepared by drop-wise addition of 1 mole equivalent of concentrated sulfuric acid to a solution of (R)-8-chloro-1-methyl-2,3,4,5-tetrahydro-1H-3-benzazepine free base in either isopropyl acetate or acetonitrile with vigorous stirring. Precipitation occurred immediately and the suspension was allowed to stir for 1 to 2 days. The resulting solid was recovered by filtration. (R)-8-Chloro-1-methyl-2,3,4,5-tetrahydro-1H-3-benzazepine bisulfate salt had an extrapolated melting onset temperature by DSC of about 162° C.

(R)-8-Chloro-1-methyl-2,3,4,5-tetrahydro-1H-3-benzazepine hemisulfate salt was prepared by the drop-wise addition of 0.5 mole equivalent of concentrated sulfuric acid to a solution of (R)-8-chloro-1-methyl-2,3,4,5-tetrahydro-1H-3-benzazepine free base in either isopropyl acetate or acetonitrile with vigorous stirring. Precipitation occurred immediately and the suspension was allowed to stir for 1 to 2 days. The resulting yellow solid was recovered by filtration. Acetone was added to the solid followed by sufficient water to cause dispersal (<5%). This mixture was slurried for 4 h and the solid was collected by centrifuge filtration (10,000 rpm for 1 min). The filtrate contained an oil droplet and the filter cake had a small amount of color at the bottom. The white upper portion of the filter cake was removed and air-dried overnight to leave the title salt as a white solid. (R)-8-Chloro-1-methyl-2,3,4,5-tetrahydro-1H-3-benzazepine hemisulfate salt had an extrapolated melting onset temperature by DSC of about 79° C.

(R)-8-Chloro-1-methyl-2,3,4,5-tetrahydro-1H-3-benzazepine mesylate salt was prepared by the dropwise addition of one equivalent of methanesulfonic acid (99.5%) to a solution of (R)-8-chloro-1-methyl-2,3,4,5-tetrahydro-1H-3-benzazepine free base in acetonitrile, or isopropyl acetate with vigorous stirring. Crystallization occurred either immediately or within 24 hours after the solution was heated to ˜60° C. and then allowed to cool to RT while stirring. (R)-8-Chloro-1-methyl-2,3,4,5-tetrahydro-1H-3-benzazepine mesylate salt had an extrapolated melting onset temperature by DSC of about 178° C.

(R)-8-Chloro-1-methyl-2,3,4,5-tetrahydro-1H-3-benzazepine hydrobromide salt hemihydrate was prepared by the dropwise addition of one equivalent of aqueous HBr (−48%) to a solution of (R)-8-chloro-1-methyl-2,3,4,5-tetrahydro-1H-3-benzazepine free base in isopropyl acetate, acetonitrile, or ethyl acetate with vigorous stirring. The product readily precipitated from the reaction in isopropyl acetate. In acetonitrile the solvent was evaporated to near dryness to obtain a solid. In ethyl acetate, seeds were added and the reaction was allowed to stir unstoppered to initiate crystallization. The reaction was then closed and stirring was continued to afford a yellow suspension. The suspension was filtered and the solid was washed with cold ethyl acetate. The resulting white solid was under nitrogen at ˜38° C., and held overnight at 25° C./75% RH. (R)-8-Chloro-1-methyl-2,3,4,5-tetrahydro-1H-3-benzazepine hydrobromide salt hemihydrate had an extrapolated dehydration onset temperature by TGA of about 72.5° C.

(R)-8-Chloro-1-methyl-2,3,4,5-tetrahydro-1H-3-benzazepine nitrate salt was prepared by dropwise addition of aqueous HNO₃ to a solution of (R)-8-chloro-1-methyl-2,3,4,5-tetrahydro-1H-3-benzazepine free base in isopropyl acetate or acetonitrile with vigorous stirring. (R)-8-Chloro-1-methyl-2,3,4,5-tetrahydro-1H-3-benzazepine nitrate salt had an extrapolated melting onset temperature by DSC of about 124° C.

(R)-8-Chloro-1-methyl-2,3,4,5-tetrahydro-1H-3-benzazepine sesqui-oxalate salt-cocrystal was prepared by addition of oxalic acid (0.5 eq.) to a solution of (R)-8-chloro-1-methyl-2,3,4,5-tetrahydro-1H-3-benzazepine in isopropyl acetate. (R)-8-Chloro-1-methyl-2,3,4,5-tetrahydro-1H-3-benzazepine sesqui-oxalate salt-cocrystal had an initial endotherm with an extrapolated onset temperature by DSC of about 105° C. and a second endotherm with an extrapolated melting onset temperature by DSC of about 111° C.

(R)-8-Chloro-1-methyl-2,3,4,5-tetrahydro-1H-3-benzazepine adipate salt was prepared by addition of adipic acid (0.5-1 eq.) in acetone to a solution of (R)-8-chloro-1-methyl-2,3,4,5-tetrahydro-1H-3-benzazepine at ˜62° C. Precipitation occurred within 5 min and the suspension was allowed to cool to ambient temperature with stirring. (R)-8-Chloro-1-methyl-2,3,4,5-tetrahydro-1H-3-benzazepine adipate salt had multiple endothermic events by DSC starting at onset temperatures between 104° C. and 107° C.

(R)-8-Chloro-1-methyl-2,3,4,5-tetrahydro-1H-3-benzazepine malonate salt was prepared by addition of malonic acid (1 eq.) to a solution of (R)-8-chloro-1-methyl-2,3,4,5-tetrahydro-1H-3-benzazepine in isopropyl acetate. (R)-8-Chloro-1-methyl-2,3,4,5-tetrahydro-1H-3-benzazepine malonate salt had an extrapolated melting onset temperature by DSC of about 143° C.

(R)-8-Chloro-1-methyl-2,3,4,5-tetrahydro-1H-3-benzazepine hemimalonate salt was prepared by addition of malonic acid (0.5 eq.) to a solution of (R)-8-chloro-1-methyl-2,3,4,5-tetrahydro-1H-3-benzazepine in isopropyl acetate. (R)-8-Chloro-1-methyl-2,3,4,5-tetrahydro-1H-3-benzazepine hemimalonate salt had an extrapolated melting onset temperature by DSC of 135-136° C.

(R)-8-Chloro-1-methyl-2,3,4,5-tetrahydro-1H-3-benzazepine glycolate salt was prepared by the addition of one equivalent of glycolic acid to a solution of (R)-8-chloro-1-methyl-2,3,4,5-tetrahydro-1H-3-benzazepine in ethyl acetate or acetone at 60° C. Glycolic acid, at 60° C., was added dropwise, in the corresponding solvent, with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine glycolate salt had an extrapolated melting onset temperature by DSC of about 138° C.

(R)-8-Chloro-1-methyl-2,3,4,5-tetrahydro-1H-3-benzazepine hemi-edisylate salt was prepared by the dropwise addition of 0.5 equivalents of aqueous 1,2-ethanedisulfonic acid dihydrate (−3.7 M) to a solution of (R)-8-chloro-1-methyl-2,3,4,5-tetrahydro-1H-3-benzazepine free base in either acetonitrile or isopropyl acetate with vigorous stirring. Immediate precipitation was observed. The solid obtained was washed with isopropyl alcohol and allowed to dry on the filter. (R)-8-Chloro-1-methyl-2,3,4,5-tetrahydro-1H-3-benzazepine hemi-edisylate salt had an extrapolated melting onset temperature by DSC of about 298° C.

(R)-8-Chloro-1-methyl-2,3,4,5-tetrahydro-1H-3-benzazepine phosphate salt was prepared by dropwise addition of ortho-phosphoric acid (85%) (0.5-1 mole equivalent) to a solution of (R)-8-chloro-1-methyl-2,3,4,5-tetrahydro-1H-3-benzazepine free base in isopropyl acetate or acetonitrile with vigorous stirring. Immediate precipitation was observed in all experiments. Initially amorphous material was slurried in acetone; initially crystalline material was slurried/ripened in n-propanol for 3 days. (R)-8-Chloro-1-methyl-2,3,4,5-tetrahydro-1H-3-benzazepine phosphate salt had an extrapolated melting onset temperature by DSC of about 208° C.

(R)-8-Chloro-1-methyl-2,3,4,5-tetrahydro-1H-3-benzazepine citrate salt hemihydrate was prepared by dropwise addition of 1 mole equivalent of citric acid in hot MeOH to a solution of (R)-8-chloro-1-methyl-2,3,4,5-tetrahydro-1H-3-benzazepine in isopropyl acetate. Precipitation occurred spontaneously. (R)-8-Chloro-1-methyl-2,3,4,5-tetrahydro-1H-3-benzazepine citrate salt hemihydrate had a dehydration onset temperature by DSC of about 80° C.

(R)-8-Chloro-1-methyl-2,3,4,5-tetrahydro-1H-3-benzazepine hemi-oxalate salt was prepared by dropwise addition of 1 mole equivalent of oxalic acid as a solid or as a solution in MeOH (˜2.5 M) to a solution of (R)-8-chloro-1-methyl-2,3,4,5-tetrahydro-1H-3-benzazepine in isopropyl acetate. (R)-8-Chloro-1-methyl-2,3,4,5-tetrahydro-1H-3-benzazepine hemi-oxalate salt had an extrapolated melting onset temperature by DSC of about 212° C.

(R)-8-Chloro-1-methyl-2,3,4,5-tetrahydro-1H-3-benzazepine succinate salt was prepared by the addition of succinic acid (0.5-1 eq.) in hot EtOH to a solution of (R)-8-chloro-1-methyl-2,3,4,5-tetrahydro-1H-3-benzazepine in isopropyl acetate. After overnight stirring, a solid was recovered by suction filtration and washed in isopropyl acetate. (R)-8-Chloro-1-methyl-2,3,4,5-tetrahydro-1H-3-benzazepine succinate salt had an extrapolated melting onset temperature by DSC of about 179.1° C.

(R)-8-Chloro-1-methyl-2,3,4,5-tetrahydro-1H-3-benzazepine oxoglutarate salt was prepared by addition of one equivalent of α-oxo-glutaric acid to a solution of (R)-8-chloro-1-methyl-2,3,4,5-tetrahydro-1H-3-benzazepine in ethyl acetate at 60° C. α-Oxo-glutaric acid in ethyl acetate at 60° C. was added dropwise with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine oxoglutarate salt had an extrapolated melting onset temperature by DSC of about 115° C.

(R)-8-Chloro-1-methyl-2,3,4,5-tetrahydro-1H-3-benzazepine oxoglutarate salt solvate was prepared by addition of a molar equivalent of α-oxo-glutaric acid to a solution of (R)-8-chloro-1-methyl-2,3,4,5-tetrahydro-1H-3-benzazepine in acetonitrile at 60° C. α-Oxo-glutaric acid in acetonitrile at 60° C. was added dropwise with vigorous stirring. Precipitation occurred immediately and the suspension was allowed to cool and stir overnight. The resulting solid was recovered by filtration and air-dried in a fume hood overnight. (R)-8-Chloro-1-methyl-2,3,4,5-tetrahydro-1H-3-benzazepine oxoglutarate salt solvate had an extrapolated desolvation onset temperature by DSC of about 91° C., and a second endotherm with an extrapolated onset temperature by DSC of about 113° C.

### Pharmaceutical Compositions

One aspect of the present invention pertains to methods for manufacturing a medicament for reducing a concentration of a catecholamine in an individual, comprising admixing a 5-HT_{2C} receptor agonist with at least one pharmaceutically acceptable excipient; wherein the 5-HT_{2C} receptor agonist has been administered to a mammal in whom a catecholamine concentration has been measured.

One aspect of the present invention pertains to methods for manufacturing a medicament for reducing a concentration of epinephrine in an individual, comprising admixing a 5-HT_{2C} receptor agonist with at least one pharmaceutically acceptable excipient; wherein the 5-HT_{2C} receptor agonist has been administered to a mammal in whom an epinephrine concentration has been measured.

One aspect of the present invention pertains to methods for manufacturing a medicament for reducing a concentration of norepinephrine in an individual, comprising admixing a 5-HT_{2C} receptor agonist with at least one pharmaceutically acceptable excipient; wherein the 5-HT_{2C} receptor agonist has been administered to a mammal in whom a norepinephrine concentration has been measured.

One aspect of the present invention pertains to methods for manufacturing a medicament for reducing a concentration of norepinephrine in an individual, comprising: measuring a norepinephrine concentration in a mammal; and admixing a 5-HT_{2C} receptor agonist with at least one pharmaceutically acceptable excipient; wherein the 5-HT_{2C} receptor agonist has been administered to the mammal.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in the mammal; and admixing the 5-HT_{2C} receptor agonist with at least one pharmaceutically acceptable excipient.

One aspect of the present invention pertains to methods for manufacturing a medicament for treating a disorder ameliorated by a reduction of a concentration of catecholamine in an individual, comprising: admixing a 5-HT_{2C} receptor agonist with at least one pharmaceutically acceptable excipient; wherein said 5-HT_{2C} receptor agonist has been administered to a mammal in whom a catecholamine concentration has been measured.

One aspect of the present invention pertains to methods for manufacturing a medicament for treating a disorder ameliorated by a reduction of a concentration of epinephrine in an individual, comprising: admixing a 5-HT_{2C} receptor agonist with at least one pharmaceutically acceptable excipient; wherein said 5-HT_{2C} receptor agonist has been administered to a mammal in whom an epinephrine concentration has been measured.

One aspect of the present invention pertains to methods for manufacturing a medicament for treating a disorder ameliorated by a reduction of a concentration of norepinephrine in an individual, comprising: admixing a 5-HT_{2C} receptor agonist with at least one pharmaceutically acceptable excipient; wherein said 5-HT_{2C} receptor agonist has been administered to a mammal in whom a norepinephrine concentration has been measured.

One aspect of the present invention pertains to methods for manufacturing a medicament for treating a disorder ameliorated by a reduction of a concentration of norepinephrine in an individual, comprising: measuring a norepinephrine concentration in a mammal; and admixing a 5-HT_{2C} receptor agonist with at least one pharmaceutically acceptable excipient; wherein the 5-HT_{2C} receptor agonist has been administered to the mammal.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in the mammal; and admixing the 5-HT_{2C} receptor agonist with at least one pharmaceutically acceptable excipient.

One aspect of the present invention pertains to methods for manufacturing a medicament for reducing a concentration of a catecholamine in an individual, comprising resynthesizing a 5-HT_{2C} receptor agonist; wherein the 5-HT_{2C} receptor agonist has been administered to a mammal in whom a catecholamine concentration has been measured.

One aspect of the present invention pertains to methods for manufacturing a medicament for reducing a concentration of epinephrine in an individual, comprising resynthesizing a 5-HT_{2C} receptor agonist; wherein the 5-HT_{2C} receptor agonist has been administered to a mammal in whom an epinephrine concentration has been measured.

One aspect of the present invention pertains to methods for manufacturing a medicament for reducing a concentration of norepinephrine in an individual, comprising resynthesizing a 5-HT_{2C} receptor agonist; wherein the 5-HT_{2C} receptor agonist has been administered to a mammal in whom a norepinephrine concentration has been measured.

One aspect of the present invention pertains to methods for manufacturing a medicament for reducing a concentration of norepinephrine in an individual, comprising: measuring a norepinephrine concentration in a mammal; and resynthesizing a 5-HT_{2C} receptor agonist; wherein the 5-HT_{2C} receptor agonist has been administered to the mammal.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in the mammal; and resynthesizing the 5-HT_{2C} receptor agonist.

One aspect of the present invention pertains to methods for manufacturing a medicament for treating a disorder ameliorated by a reduction of a concentration of a catecholamine in an individual, comprising resynthesizing a 5-HT_{2C} receptor agonist; wherein the 5-HT_{2C} receptor agonist has been administered to a mammal in whom a catecholamine concentration has been measured.

One aspect of the present invention pertains to methods for manufacturing a medicament for treating a disorder ameliorated by a reduction of a concentration of epinephrine in an individual, comprising resynthesizing a 5-HT_{2C} receptor agonist; wherein the 5-HT_{2C} receptor agonist has been administered to a mammal in whom an epinephrine concentration has been measured.

One aspect of the present invention pertains to methods for manufacturing a medicament for treating a disorder ameliorated by a reduction of a concentration of norepinephrine in an individual, comprising resynthesizing a 5-HT_{2C} receptor agonist; wherein the 5-HT_{2C} receptor agonist has been administered to a mammal in whom a norepinephrine concentration has been measured.

One aspect of the present invention pertains to methods for manufacturing a medicament for treating a disorder ameliorated by a reduction of a concentration of norepinephrine in an individual, comprising: measuring a norepinephrine concentration in a mammal; and resynthesizing a 5-HT_{2C} receptor agonist; wherein the 5-HT_{2C} receptor agonist has been administered to the mammal.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in the mammal; and resynthesizing the 5-HT_{2C} receptor agonist.

One aspect of the present invention pertains to methods for manufacturing a medicament for reducing a concentration of a catecholamine in an individual, comprising admixing a 5-HT_{2C} receptor agonist with at least one pharmaceutically acceptable excipient; wherein the 5-HT_{2C} receptor agonist has been administered to a mammal in whom a catecholamine concentration has been measured, and wherein the catecholamine concentration in the mammal has been reduced.

One aspect of the present invention pertains to methods for manufacturing a medicament for reducing a concentration of epinephrine in an individual, comprising admixing a 5-HT_{2C} receptor agonist with at least one pharmaceutically acceptable excipient; wherein the 5-HT_{2C} receptor agonist has been administered to a mammal in whom an epinephrine concentration has been measured, and wherein the epinephrine concentration in the mammal has been reduced.

One aspect of the present invention pertains to methods for manufacturing a medicament for reducing a concentration of norepinephrine in an individual, comprising admixing a 5-HT_{2C} receptor agonist with at least one pharmaceutically acceptable excipient;

wherein the 5-HT_{2C} receptor agonist has been administered to a mammal in whom a norepinephrine concentration has been measured, and wherein the norepinephrine concentration in the mammal has been reduced.

One aspect of the present invention pertains to methods for manufacturing a medicament for reducing a concentration of norepinephrine in an individual, comprising: measuring a norepinephrine concentration in a mammal; and admixing a 5-HT_{2C} receptor agonist with at least one pharmaceutically acceptable excipient; wherein the 5-HT_{2C} receptor agonist has been administered to the mammal, and wherein the norepinephrine concentration in the mammal has been reduced.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in the mammal; and admixing the 5-HT_{2C} receptor agonist with at least one pharmaceutically acceptable excipient; wherein the norepinephrine concentration in the mammal has been reduced; wherein the norepinephrine concentration in the mammal has been reduced.

One aspect of the present invention pertains to methods for manufacturing a medicament for treating a disorder ameliorated by a reduction of a concentration of catecholamine in an individual, comprising: admixing a 5-HT_{2C} receptor agonist with at least one pharmaceutically acceptable excipient; wherein said 5-HT_{2C} receptor agonist has been administered to a mammal in whom a catecholamine concentration has been measured, and wherein the catecholamine concentration in the mammal has been reduced.

One aspect of the present invention pertains to methods for manufacturing a medicament for treating a disorder ameliorated by a reduction of a concentration of epinephrine in an individual, comprising: admixing a 5-HT_{2C} receptor agonist with at least one pharmaceutically acceptable excipient; wherein said 5-HT_{2C} receptor agonist has been administered to a mammal in whom an epinephrine concentration has been measured, and wherein the epinephrine concentration in the mammal has been reduced.

One aspect of the present invention pertains to methods for manufacturing a medicament for treating a disorder ameliorated by a reduction of a concentration of norepinephrine in an individual, comprising: admixing a 5-HT_{2C} receptor agonist with at least one pharmaceutically acceptable excipient; wherein said 5-HT_{2C} receptor agonist has been administered to a mammal in whom a norepinephrine concentration has been measured, and wherein the norepinephrine concentration in the mammal has been reduced.

One aspect of the present invention pertains to methods for manufacturing a medicament for treating a disorder ameliorated by a reduction of a concentration of norepinephrine in an individual, comprising: measuring a norepinephrine concentration in a mammal; and admixing a 5-HT_{2C} receptor agonist with at least one pharmaceutically acceptable excipient; wherein the 5-HT_{2C} receptor agonist has been administered to the mammal, and wherein the norepinephrine concentration in the mammal has been reduced.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in the mammal; and admixing the 5-HT_{2C} receptor agonist with at least one pharmaceutically acceptable excipient; wherein the norepinephrine concentration in the mammal has been reduced.

One aspect of the present invention pertains to methods for manufacturing a medicament for reducing a concentration of a catecholamine in an individual, comprising resynthesizing a 5-HT_{2C} receptor agonist; wherein the 5-HT_{2C} receptor agonist has been administered to a mammal in whom a catecholamine concentration has been measured, and wherein the catecholamine concentration in the mammal has been reduced.

One aspect of the present invention pertains to methods for manufacturing a medicament for reducing a concentration of epinephrine in an individual, comprising resynthesizing a 5-HT_{2C} receptor agonist; wherein the 5-HT_{2C} receptor agonist has been administered to a mammal in whom an epinephrine concentration has been measured, and wherein the epinephrine concentration in the mammal has been reduced.

One aspect of the present invention pertains to methods for manufacturing a medicament for reducing a concentration of norepinephrine in an individual, comprising resynthesizing a 5-HT_{2C} receptor agonist; wherein the 5-HT_{2C} receptor agonist has been administered to a mammal in whom a norepinephrine concentration has been measured, and wherein the norepinephrine concentration in the mammal has been reduced.

One aspect of the present invention pertains to methods for manufacturing a medicament for reducing a concentration of norepinephrine in an individual, comprising: measuring a norepinephrine concentration in a mammal; and resynthesizing a 5-HT_{2C} receptor agonist; wherein the 5-HT_{2C} receptor agonist has been administered to the mammal, and wherein the norepinephrine concentration in the mammal has been reduced.

One aspect of the present invention pertains to methods for manufacturing a medicament for reducing a concentration of norepinephrine in an individual, comprising: administering a 5-HT_{2C} receptor agonist to a mammal; measuring a norepinephrine concentration in the mammal; and resynthesizing the 5-HT_{2C} receptor agonist; wherein the norepinephrine concentration in the mammal has been reduced.

One aspect of the present invention pertains to methods for manufacturing a medicament for treating a disorder ameliorated by a reduction of a concentration of a catecholamine in an individual, comprising resynthesizing a 5-HT_{2C} receptor agonist; wherein the 5-HT_{2C} receptor agonist has been administered to a mammal in whom a catecholamine concentration has been measured, and wherein the catecholamine concentration in the mammal has been reduced.

One aspect of the present invention pertains to methods for manufacturing a medicament for treating a disorder ameliorated by a reduction of a concentration of epinephrine in an individual, comprising resynthesizing a 5-HT_{2C} receptor agonist; wherein the 5-HT_{2C} receptor agonist has been administered to a mammal in whom an epinephrine concentration has been measured, and wherein the epinephrine concentration in the mammal has been reduced.

One aspect of the present invention pertains to methods for manufacturing a medicament for treating a disorder ameliorated by a reduction of a concentration of norepinephrine in an individual, comprising resynthesizing a 5-HT_{2C} receptor agonist; wherein the 5-HT_{2C} receptor agonist has been administered to a mammal in whom a norepinephrine concentration has been measured, and wherein the norepinephrine concentration in the mammal has been reduced.

One aspect of the present invention pertains to methods for manufacturing a medicament for treating a disorder ameliorated by a reduction of a concentration of norepinephrine in an individual, comprising: measuring a norepinephrine concentration in a mammal; and resynthesizing a 5-HT_{2C} receptor agonist; wherein the 5-HT_{2C} receptor agonist has been administered to the mammal, and wherein the norepinephrine concentration in the mammal has been reduced.

One aspect of the present invention pertains to methods for manufacturing a medicament for treating a disorder ameliorated by a reduction of a concentration of norepinephrine in an individual, comprising: administering a 5-HT_{2C} receptor agonist to a mammal; measuring a norepinephrine concentration in the mammal; and resynthesizing the 5-HT_{2C} receptor agonist; wherein the norepinephrine concentration in the mammal has been reduced.

In some embodiments, the norepinephrine concentration in the mammal was reduced within 56 days of administering the first dose of the 5-HT_{2C} receptor agonist to the mammal.

In some embodiments, the norepinephrine concentration in the mammal was reduced within 7 days of administering the first dose of the 5-HT_{2C} receptor agonist to the mammal.

In some embodiments, the norepinephrine concentration in the mammal was reduced independently of concomitant weight-loss in the mammal.

In some embodiments, the mammal did not lose a substantial amount of weight during the period in which the norepinephrine concentration in the mammal was reduced.

In some embodiments, the mammal did lose a substantial amount of weight during the period in which the norepinephrine concentration in the mammal was reduced and the norepinephrine concentration in the mammal was reduced by more than would be expected solely as a result of the mammal loosing the substantial amount of weight.

In some embodiments, the norepinephrine concentration is a urine norepinephrine concentration.

In some embodiments, the norepinephrine concentration is a blood norepinephrine concentration.

In some embodiments, the norepinephrine concentration is a plasma norepinephrine concentration.

In some embodiments, the norepinephrine concentration is a brain norepinephrine concentration.

In some embodiments, the norepinephrine concentration in the mammal was reduced by least about 10% lower than baseline.

In some embodiments, the norepinephrine concentration in the mammal was reduced by least about 20% lower than baseline.

In some embodiments, the norepinephrine concentration in the mammal was reduced by least about 30% lower than baseline.

In some embodiments, the norepinephrine concentration in the mammal was reduced by least about 40% lower than baseline.

In some embodiments, the norepinephrine concentration in the mammal was reduced by least about 50% lower than baseline.

In some embodiments, the 5-HT_{2C} receptor agonist is a small molecule.

In some embodiments, the 5-HT_{2C} receptor agonist is orally bioavailable.

In some embodiments, the selective 5-HT_{2C} receptor agonist is selected from 8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is selected from (R)-8-chloro-1-methyl-2,3,4,5-tetrahydro-1H-3-benzazepine, and pharmaceutically acceptable salts, solvates, and hydrates thereof.

In some embodiments, the selective 5-HT_{2C} receptor agonist is selected from (R)-8-chloro-1-methyl-2,3,4,5-tetrahydro-1H-3-benzazepine hydrochloride salt, and pharmaceutically acceptable solvates and hydrates thereof.

In some embodiments, the selective 5-HT_{2C} receptor agonist is (R)-8-chloro-1-methyl-2,3,4,5-tetrahydro-1H-3-benzazepine hydrochloride salt hemihydrate.

One aspect of the present invention pertains to methods for manufacturing a medicament for reducing a concentration of norepinephrine in an individual, or for treating a disorder ameliorated by reducing a concentration of norepinephrine in an individual comprising admixing a 5-HT_{2C} receptor agonist with at least one pharmaceutically acceptable excipient; wherein the 5-HT_{2C} receptor agonist has been administered to a mammal in whom a norepinephrine concentration has been measured.

One aspect of the present invention pertains to methods for manufacturing a medicament for reducing a concentration of norepinephrine in an individual, or for treating a disorder ameliorated by reducing a concentration of norepinephrine in an individual comprising: measuring a norepinephrine concentration in a mammal; and admixing a 5-HT_{2C} receptor agonist with at least one pharmaceutically acceptable excipient; wherein the 5-HT_{2C} receptor agonist has been administered to the mammal.

One aspect of the present invention pertains to methods for manufacturing a medicament for reducing a concentration of norepinephrine in an individual, or for treating a disorder ameliorated by reducing a concentration of norepinephrine in an individual comprising: administering a 5-HT_{2C} receptor agonist to a mammal; measuring a norepinephrine concentration in the mammal; and admixing the 5-HT_{2C} receptor agonist with at least one pharmaceutically acceptable excipient.

One aspect of the present invention pertains to methods for manufacturing a medicament for reducing a concentration of norepinephrine in an individual, or for treating a disorder ameliorated by reducing a concentration of norepinephrine in an individual comprising resynthesizing a 5-HT_{2C} receptor agonist; wherein the 5-HT_{2C} receptor agonist has been administered to a mammal in whom a norepinephrine concentration has been measured.

One aspect of the present invention pertains to methods for manufacturing a medicament for reducing a concentration of norepinephrine in an individual, or for treating a disorder ameliorated by reducing a concentration of norepinephrine in an individual comprising: measuring a norepinephrine concentration in a mammal; and resynthesizing a 5-HT_{2C} receptor agonist; wherein the 5-HT_{2C} receptor agonist has been administered to the mammal.

One aspect of the present invention pertains to methods for manufacturing a medicament for reducing a concentration of norepinephrine in an individual, or for treating a disorder ameliorated by reducing a concentration of norepinephrine in an individual comprising: administering a 5-HT_{2C} receptor agonist to a mammal; measuring a norepinephrine concentration in the mammal; and resynthesizing the 5-HT_{2C} receptor agonist.

In some embodiments, the disorder is selected from: hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

Medicaments, or pharmaceutical compositions, may be prepared by any suitable method, typically by uniformly mixing the active compound(s) with liquids or finely divided solid carriers, or both, in the required proportions and then, if necessary, forming the resulting mixture into a desired shape.

Compounds described herein and solvates, hydrates and physiologically functional derivatives thereof can be used as active ingredients in pharmaceutical compositions, specifically as 5-HT_{2C} receptor agonists for treating disorders ameliorated by the reduction of an individual's norepinephrine level. The term “active ingredient” as defined in the context of a “pharmaceutical composition” is intended to mean a component of a pharmaceutical composition that provides the primary pharmacological effect, as opposed to an “inactive ingredient” which would generally be recognized as providing no pharmaceutical benefit.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound described herein can be formulated into a pharmaceutical composition using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that a compound described herein may be administered as a raw or pure chemical for use in method treatment of the present invention, it is preferable however to present the active pharmaceutical ingredient as a pharmaceutical formulation or composition further comprising a pharmaceutically acceptable carrier.

The invention thus further provides methods of using pharmaceutical formulations comprising a compound described herein or a pharmaceutically acceptable salt, solvate, hydrate or derivative thereof, together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compositions described herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compositions or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

The dose when using the compositions described herein can vary within wide limits and as is customary and is known to the physician, it is to be tailored to the individual conditions in each individual case. It depends, for example, on the nature and severity of the illness to be treated, on the condition of the patient, on the active pharmaceutical ingredient employed or on whether an acute or chronic disease state is treated or prophylaxis is conducted or on whether further active compositions are administered in addition to the compositions described herein. Representative doses include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example two, three or four doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular active pharmaceutical ingredient employed, whether a drug delivery system is utilized, on whether an acute or chronic disease state is being treated or prophylaxis is conducted or on whether further active compositions are administered in addition to the compositions of the present invention and as part of a drug combination. The dosage regimen for treating a disease condition with the compositions and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example two-, three- or four-part administrations. If appropriate, depending on individual behavior, it may be necessary to deviate upward or downward from the daily dose indicated.

The compositions described herein can be administrated in a wide variety of oral and parenteral dosage forms. It will be obvious to those skilled in the art that the following dosage forms may comprise an active pharmaceutical ingredient of the invention.

For preparing pharmaceutical compositions from the compounds described herein, the pharmaceutically acceptable carriers can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. In tablets, the active component is mixed with the carrier having the necessary binding capacity in suitable proportions and compacted to the desire shape and size. The powders and tablets may contain varying percentage amounts of the active pharmaceutical ingredient. A representative amount in a powder or tablet may contain from 0.5 to about 90 percent of the active pharmaceutical ingredient; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like.

Preparing pharmaceutical compositions optionally includes the formulation of the active pharmaceutical ingredient with an encapsulating material as a carrier thus providing a capsule in which the active component, with or without further carriers, is surrounded by and in association with a carrier.

Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions described herein may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compositions described herein may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compositions of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the pharmaceutical compositions of the present invention as an aerosol can be prepared by processes well known to the person skilled in the art. For their preparation, for example, solutions or dispersions of the pharmaceutical compositions of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the active pharmaceutical ingredient will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the active pharmaceutical ingredient in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds described herein optionally comprise pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds described herein which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, arginine, L-arginine, tris(trihydroxymethyl)aminomethane, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The active pharmaceutical ingredients described herein may form solvates with standard low molecular weight solvents using methods known to the skilled artisan.

Active pharmaceutical ingredients described herein can be converted to “pro-drugs.” The term “pro-drugs” refers to compounds that have been modified with specific chemical groups known in the art and when administered into an individual these groups undergo biotransformation to give the parent compound. Pro-drugs can thus be viewed as active pharmaceutical ingredients containing one or more specialized non-toxic protective groups used in a transient manner to alter or to eliminate a property of the active pharmaceutical ingredient. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Pharmaceutical compositions for “combination-therapy” may be prepared by admixing at least two pharmaceutical agents described herein and a pharmaceutically acceptable carrier.

It is noted that when selective 5-HT_{2C} receptor agonists are utilized as active ingredients in pharmaceutical compositions, these are not intended for use only in humans, but in other non-human animals as well. Indeed, recent advances in the area of animal health-care mandate that consideration be given for the use of active agents, such as selective 5-HT_{2C} receptor agonists, for the treatment of disorders ameliorated by reduction of norepinephrine level in companionship animals (e.g., cats, dogs, etc.) and in livestock animals (e.g., cows, chickens, fish, etc.) Those of ordinary skill in the art are readily credited with understanding the utility of such active pharmaceutical ingredients in such settings.

### Hydrates and Solvates

It is understood that when the phrase “pharmaceutically acceptable salts, solvates, and hydrates” or the phrase “pharmaceutically acceptable salt, solvate, or hydrate” is used when referring to the 5-HT_{2C} receptor agonists described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used in reference to salts described herein, it embraces pharmaceutically acceptable solvates and/or hydrates of such salts.

Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Hydrates and solvates can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Isotopes

Isotopes include those atoms having the same atomic number but different mass numbers. It is understood that the 5-HT_{2C} receptor agonists described herein include 5-HT_{2C} receptor agonists containing any of the isotopes of their constituent atoms.

One such example is the replacement of an atom that is the most naturally abundant isotope, such as ¹H or ¹²C, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I and ¹³¹I.

Compositions, including pharmaceutical compositions, for use in treating one or more of the disorders and conditions described herein, comprising any of the 5-HT_{2C} receptor agonists described herein, can be prepared wherein the naturally occurring distribution of the isotopes in the composition is perturbed. Compositions, including pharmaceutical compositions, for use in treating one or more of the disorders and conditions described herein, comprising any of the 5-HT_{2C} receptor agonists described herein, can be prepared wherein the 5-HT_{2C} receptor agonist is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

### Polymorphism

Polymorphism is the ability of a substance to exist as two or more crystalline phases that have different arrangements and/or conformations of the molecules in the crystal lattice. Polymorphs show the same properties in the liquid or gaseous state but they may behave differently in the solid state. Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another. It is understood that the 5-HT_{2C} receptor agonists described herein include all polymorphs and pseudopolymorphs thereof.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

Recently, polymorph screens of 245 compounds revealed that about 90% of them exhibited multiple solid forms. Overall, approximately half the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026.)

### Indications

### 1. Hypercatecholaminemia

Hypernorepinephrinemia is characterized by increased levels of norepinephrine in the body, as measured peripherally. According to the National Institutes of Health (NIH), the normal values for norepinephrine are 15-80 μg/24 h in urine, and 0-600 pg/mL in blood. Reduction of norepinephrine level in hypernorepinephrinemic intervals is achieved by administering a therapeutically effective amount of a 5-HT_{2C} receptor agonist.

The NIH reports normal values are 0.5-20 μcg/24 h for urine epinephrine, and 14-110 μcg/24 h for total urine catecholamines.

### 2. Diabetes

Diabetes mellitus is a serious disease afflicting over 100 million people worldwide. In the United States, there are more than 12 million diabetics, with 600,000 new cases diagnosed each year. Type I diabetes involves autoimmune destruction of insulin secreting pancreatic beta cells. The sympathetic nervous system innervates lymphoid organs and potentiates immune responses via adrenoceptors. Norepinephrine mediates destruction of beta cells as an extreme form of downregulation of insulin production through activation of T cells. Type 2 diabetes is characterized by hyperglycemia, hyperinsulinemia, and insulin resistance. Sustained inhibition of insulin production through increased stimulation of adrenoceptors on pancreatic beta cells by norepinephrine results in hyperglycemia and affects insulin resistance. Type 1 and type 2 diabetes mellitus can be treated by lowering an individual's norepinephrine level. Accordingly, type 1 and type 2 diabetes can be treated by administering a 5-HT_{2C} receptor agonist. See, e.g., Ni X-P., et al., Evidence for a noradrenergic mechanism causing hypertension and abnormal glucose metabolism in rats with relative deficiency of γ-melanocyte-stimulating hormone, Exp. Physiol. (2009); Penesova A., et al., The Role of Norepinephrine and Insulin Resistance in an Early Stage of Hypertension, Ann. NY Acad. Sci. (2008) 1148:490-494; LeRoith D., et al., Mouse models created to study the physiology of Type 2 diabetes, International Journal of Biochemistry and Cell Biology (2006) 38:904-912; Nandi A., et al., Mouse Models of Insulin Resistance, Physiol. Rev. (2004) 84:623-647; Rees D. A., et al., Animal models of diabetes mellitus, Diabet. Med. (2005) 22:359-370; Postic C., et al., Mouse models of insulin resistance and type 2 diabetes, Ann. Endocrinol. (2004) 65:51-59; Chen D., et al., Development and application of rodent models for type 2 diabetes, Diabetes, Obesity and Metabolism (2005) 7:307-317; Baddinger S. B., et al., From Mice to Men: Insights into the Insulin Resistance Syndromes, Annu. Rev. Physiol. (2006) 68:123-58; Yatabe M. S., Salt sensitivity is associated with insulin resistance, sympathetic overactivity, and decreased suppression of circulating rennin activity in lean patients with essential hypertension, Am. J. Clin. Nutr. (2010) 92:77-82; Vasudevan A. R., et al., Insulin resistance syndrome, Minerva Endocrinol (2005) 30:101-19.

### 3. Cardiomyopathy

Cardiomyopathy is a weakening of the heart muscle or a change in heart muscle structure. Common types of cardiomyopathy include dilated cardiomyopathy, restrictive cardiomyopathy and Hypertrophic cardiomyopathy in which the heart muscle becomes thick. Elevated circulating norepinepherine exerts prohypertrophic effects on the myocardial tissue as well as on the peripheral vascular system. Grassi G., et al., Essential hypertension and the sympathetic nervous system, Neurol. Sci. (2008) 29:S33-S36. Norepinepherine-induced hypertension can cause left ventricular hypertrophy and adverse myocardial remodeling. Norepinepherine-induced chronic tachycardia causes tachycardia-mediated cardiomyopathy. Joynt K. E., Paragangliomas, Cardiology in Rev. (2009) 17:159-164. Elevated norepinepherine can cause cardiomyocyte hypertrophy in post-myocardial infarction remodeling. Bonnefont-Rousselot, D. et al. Catecholamine effects on cardiac remodelling, oxidative stress and fibrosis in experimental heart failure. Redox Report (2002), 7(3), 145-151. Furthermore, Mobine et al. investigated the development of dilated cardiomyopathy in the presence of a pheochromocytoma and found that norepinephrine is necessary to induce cardiomyopathy. 5-HT_{2C} receptor agonists, which reduce norepinephrine levels can be used in the treatment of cardiomyopathy. See also: Mobine H. R., et al., Pheochromocytoma-Induced Cardiomyopathy is Modulated by the Synergistic Effects of Cell-Secreted Factors, Circ. Heart Fail. (2009) 2:121-128; Coons J. C., et al., Takotsubo cardiomyopathy, Am. J. Health-Syst. Pharm. (2009) 66:562-566; Nef H. M., et al., Mechanisms of stress (Takotsubo) cardiomyopathy, Nat. Rev. Cardiol. (2010) 7:187-193; Wang L. et al., Narrative Review: Harnessing Molecular Genetics for the Diagnosis and Management of Hypertrophic Cardiomyopathy, Ann. Intern. Med. (2010) 152:513-520; Shephard R., et al., Role of Animal Models in HCM Research, J. of Cardiovasc. Trans. Res. (2009) 2:471-482.

### 4. Elevated Heart Rate

In patients with hypertension, obesity, or heart failure there is a significant relationship between the elevated levels of plasma norepinephrine and heart rate values. Pharmaceutical intervention with 5-HT_{2C} receptor agonists, which lower plasma norepinephrine levels is thus useful for treating elevated heart rate. Grassi G., et al., Heart Rate, Sympathetic Cardiovascular Influences, and The Metabolic Syndrome, Prog. Cardiovasc. Dis. (2009) 52:31-37; Grassi G., et al., Heart rate, sympathetic cardiovascular influences, and the metabolic syndrome, Prog. Cardiovasc. Dis. (2009) 52:31-7; Villareal R. P., et al., Heart Rate Variability and Cardiovascular Mortality, Current Atherosclerosis Reports (2002) 4:120-127.

### 5. Vasoconstriction

Vasoconstriction, which plays a role in hypertension and erectile dysfunction, normally occurs when sympathetic nerves release norepinephrine. 5-HT_{2C} receptor agonists, which decrease norepinephrine levels can be used to reduce vasoconstriction, and accordingly can be used to treat, for example, hypertension, erectile dysfunction and acute pulmonary vasoconstriction. Kulik T. J., Pathophysiology of acute pulmonary vasoconstriction, Pediatr. Crit. Care Med. (2010) 11:S10-S14; Joyner M. J., et al., The Catecholamines Strike Back—What NO Does Not Do—, Circ. J. (2009) 73:1783-1792).

### 6. Hypertension

In lean hypertensive subjects, the circulating levels of norepinephrine are significantly increased as compared to those found in age-matched lean normotensive controls. Elevated plasma levels of norepinephrine are due to an increase in sympathetic discharge. 5-HT_{2C} receptor agonists, which decrease norepinephrine levels may be used to treat hypertension. Neuschmelting V., et al., Norepinephrine-induced hypertension dilates vasospastic basilar artery after subarachnoid haemorrhage in rabbits, Acta Neurochir. (2009) 151:487-493; Rocchini A. P., et al., Serial Changes in Norepinephrine Kinetics Associated With Feeding Dogs a High-Fat Diet, J. Clin. Hypertens. (2010) 12:117-124; Palatini P., et al., The Role of Cardiac Autonomic Function in Hypertension and Cardiovascular Disease, Current Hypertension Reports (2009) 11:199-205; Grassi G., Assessment of Sympathetic Cardiovascular Drive in Human Hypertension, Hypertension (2009) 54:690-697; Grassi G., et al., Essential hypertension and the sympathetic nervous system, Neurol. Sci. (2008) 29:S33-S36; Kaye D. M., et al., Autonomic Control of the Aging Heart, Neuromol. Med. (2008) 10:179-186.

### 7. Heart Failure

Patients with congestive heart failure have high plasma norepinephrine levels due to activation of sympathetic nervous system. Recent studies have shown that the administration of β-adrenergic receptor blockers improve cardiac performance and reduce cardiac mortality. Administration of 5-HT_{2C} receptor agonists, which reduce norepinephrine levels, can also improve cardiac performance and reduce cardiac mortality. Kimura K., et al., Norepinephrine-induced nerve growth factor depletion causes cardiac sympathetic denervation in severe heart failure, Auton. Neurosci. Basic Clin. (2010); Colucci W. S., The Effects of Norepinephrine on Myocardial Biology Implications for the Therapy of Heart Failure, Clin. Cardiol. (1998) 21(Suppl I):I-20-124;

8. Cardiac Dysfunction after Stroke

Stroke can lead to cardiac dysfunction via excess norepinephrine release. Stroke-induced cardiac dysfunction can be mitigated by administering a 5-HT_{2C} receptor agonist. Min J., et al., Cardiac Dysfunction After Left Permanent Cerebral Focal Ischemia, Stroke (2009) 40:2560-2563.

### 9. Cardiac Arrhythmia

Norepinephrine exacerbates several potential mechanisms of arrhythmia elicited by reperfusion in a model of cardiac ischemia and reperfusion. Lukas, A. and Ferrier, G. R., Arrhythmic effects of norepinephrine in a model of cardiac ischemia and reperfusion, Canadian Journal of Physiology and Pharmacology (1989), 67(7), 765-71. Hearts from animals following acute experimental subarachnoid hemorrhage exhibit enhanced sensitivity to norepinephrine infusion and sympathetic nerve stimulation, and are more prone to develop arrhythmias. Lambert, E. et al., Cardiac response to norepinephrine and sympathetic nerve stimulation following experimental subarachnoid hemorrhage, Journal of the Neurological Sciences (2002), 198(1-2), 43-50. Administration of 5-HT_{2C} receptor agonists, which reduce norepinephrine levels, can be used to prevent cardiac arrhythmias.

### 10. Metabolic Syndrome

Metabolic syndrome refers to a collection of disorders including type 2 diabetes mellitus, impaired fasting glucose, glucose intolerance, impaired glucose uptake, dyslipidemia and hypertension, each of which is exacerbated by norepinephrine. Metabolic syndrome can be treated by administering a 5-HT_{2C} receptor agonist which reduces norepinephrine levels. Fonseca V. A., The metabolic syndrome, hyperlipidemia, and insulin resistance, Clinical cornerstone (2005), 7(2-3), 61-72; Kennedy A. J. et al., Mouse models of the metabolic syndrome, Disease models & mechanisms (2010), 3(3-4), 156-66; Boehm O. and Claudi-Boehm, S., The metabolic syndrome, Scandinavian Journal of Clinical and Laboratory Investigation, Supplement (2005), 65(240, Diabetes Mellitus and Cardiovascular Disease), 3-13; Reaven P., Metabolic syndrome, Journal of insurance medicine (New York, N.Y.) (2004), 36(2), 132-42; Gogia A. and Agarwal P. K., Metabolic syndrome, Indian Journal of Medical Sciences (2006), 60(2), 72-81; Mueller-Wieland, D. and Kotzka, J., Correction of insulin resistance and the metabolic syndrome, Handbook of Experimental Pharmacology (2005), 170 (Atherosclerosis), 591-617.

### 11. Abnormal Lipid Metabolism

Dopamine is converted to norepinephrine by dopamine-β-hydroxylase (DBH). Inhibitors of dopamine-beta-hydroxylase (DBH) are reported (WO2009/097416) to be useful for a variety of clinical purposes including treating diseases or conditions which are positively affected by increased dopamine and/or by decreased norepinephrine, such as, abnormal lipid metabolism. 5-HT_{2C} receptor agonists that lower norepinephrine levels are useful in treating these diseases and conditions.

### 12. Hyperthermia

Hyperthermia results from a severe, unregulated rise in core body temperature. Facultative thermogenesis is mediated by norepinephrine-induced activation of skeletal muscle uncoupling protein 3. Sprague, J. E. et al., Roles of norepinephrine, free fatty acids, thyroid status, and skeletal muscle uncoupling protein 3 expression in sympathomimetic-induced thermogenesis. Journal of Pharmacology and Experimental Therapeutics (2007), 320(1), 274-280. Hypothermia can be treated by administering a 5-HT_{2C} receptor agonist, which lowers norepinephrine levels

### 13. Cushing Syndrome

Amos and Roberts have reported that a norepinephrine producing right adrenal pheochromocytoma was associated with bilateral adrenal hyperplasia and clinically and biochemically evident Cushing syndrome. Amos A. M. and McRoberts J. W. Cushing's syndrome associated with a pheochromocytoma. Urology (1998), 52(2), 331-5. Administration of 5-HT_{2C} receptor agonists, which reduce norepinephrine levels, can be used to treat Cushing Syndrome.

### 14. Pheochromocytoma

Pheochromocytomas are tumors that secrete epinephrine, norepinepherine and dopamine, causing debilitating symptoms and a poor quality of life. The debilitating symptoms of excess norepinepherine secretion from Pheochromocytomas can be ameliorated by administering a 5-HT_{2C} receptor agonist. Surgical removal of pheochromocytomas causes potentially lethal swings in blood pressure, which can be mitigated by contemporaneous administration of a 5-HT_{2C} receptor agonist. In some embodiments, such mitigation is achieved by administering a single dose of a 5-HT_{2C} receptor agonist. In some embodiments, such mitigation is achieved by short-term use of a 5-HT_{2C} receptor agonist. In some embodiments, such mitigation is achieved by acute use of a 5-HT_{2C} receptor agonist. Rossi A. P., et al., Recurrent Takotsubo Cardiomyopathy Associated With Pheochromocytoma, Endocr. Pract. (2009) 15:560-562; Adler J. T., et al., Pheochromocytoma: Current Approaches and Future Directions, The Oncologist (2008) 13:779-793; Tsai C-C., et al., Stimulatory effect of trans-cinnamaldehyde on norephinephrine secretion in cultured pheochromocytoma (PC-12) cells, Acta Pharmacol. Sin. (2000) 21:1174-1178; Mobine H. R., et al., Pheochromocytoma-Induced Cardiomyopathy is Modulated by the Synergistic Effects of Cell-Secreted Factors, Circ. Heart Fail. (2009) 2:121-128; Mobine H. R., et al., Encapsulated Pheochromocytoma Cells Secrete Potent Noncatecholamine Factors, Tissue Engineering (2009) 15:1719-1728; Cook L. K., Pheochromocytoma, American Journal of Nursing (2009) 109:50-53; Joynt K. E., Paragangliomas, Cardiology in Rev. (2009) 17:159-164; Plouin P-F., Pheochromocytomas and secreting paragangliomas, Orphanet Journal of Rare Diseases (2006) I:49.

### 15. Epilepsy

Too high or too low an extra cellular concentration of norepinephrine is proconvulsant. A mid-range brain concentration is most healthy. Administering a 5-HT_{2C} receptor agonist to an individual with too high an extra cellular concentration of norepinephrine is useful in the treatment of epilepsy. Fitzgerald P. J., Is elevated norepinephrine an etiological factor in some cases of epilepsy?, Seizure (2010) July; 19(6):311-8.

### 16. Obstructive Sleep Apnea

Patients with obstructive sleep apnea (OSA) are susceptible to transient increases in sympathetic nervous activity and hypertensive apneics maintain increased sympathetic nervous release of norepinephrine in the daytime. Administering a 5-HT_{2C} receptor agonist to an individual with OSA is useful in treating the symptoms of OSA. Ziegler M. G. et al., Sleep apnea, norepinephrine-release rate, and daytime hypertension, Sleep (1997), 20(3), 224-31; Kaditis, A. G. et al., Urine concentrations of cysteinyl leukotrienes in children with obstructive sleep-disordered breathing, Chest (2009), 135(6), 1496-1501; Vgontzas A. N. et al., Sleep apnea is a manifestation of the metabolic syndrome, Sleep medicine reviews (2005), 9(3), 211-24; Snow A. B. et al., Catecholamine alterations in pediatric obstructive sleep apnea: effect of obesity, Pediatric pulmonology (2009), 44(6), 559-67; Hersi A. S., Obstructive sleep apnea and cardiac arrhythmias, Annals of thoracic medicine (2010), 5(1), 10-7; Bopparaju and Surani, Sleep and diabetes, International journal of endocrinology (2010), 2010 759509.

### 17. Insomnia

A common thread in the mechanism of action of many sleep pharmacotherapies is norepinephrine. For instance, while not typically prescribed for insomnia or other sleep disorders, many medications that suppress the adrenergic system. Norepinephrine may also be important for the sedative effects of antihistamines because norepinephrine and histamine have reciprocal feedback and enhance each other's release. norepinephrine is also involved in the synthesis of melatonin, and thus may help to regulate the circadian as well as the homeostatic sleep processes. 5-HT_{2C} receptor agonists, which reduce norepinephrine levels, can be used to treat sleep disorders, including insomnia. Mitchell, H. A. and Weinshenker, D., Good night and good luck: Norepinephrine in sleep pharmacology. Biochemical Pharmacology (2010), 79(6), 801-809.

### 18. Glaucoma

Glaucoma comprises a family of diseases that involve damage to the optic nerve, possibly resulting in blindness. Elevated intraocular pressure is an important risk factor. Open-angle glaucoma involves decreased drainage and increased secretion of ocular fluid. Norepinephrine regulates such drainage and secretion via adrenoceptors. 5-HT_{2C} receptor agonists, which reduce norepinephrine levels, can be used to treat glaucoma. Fitzgerald P. J., Is elevated noradrenalin an aetiological factor in a number of diseases?, Autonomic and Autacoid Pharmacology (2009) 29:141-156.

### 19. Osteoarthritis and Rheumatoid Arthritis

Arthritis comprises a group of diseases characterized by pain, inflammation and damage to the body's joints. Osteoarthritis involves inflammation and degeneration of the joints, with a variety of hypothesized aetiological mechanisms such as repetitive mechanical trauma or simply ageing. Rheumatoid arthritis is thought to be an autoimmune disorder in which the body's own immune system targets the joints, resulting in damage and inflammation. Norepinephrine affects inflammation in arthritis through its effects on other signaling molecules such as cytokines. 5-HT_{2C} receptor agonists, which reduce norepinephrine levels, can be used to treat inflammatory diseases such as osteoarthritis and rheumatoid arthritis. Fitzgerald P. J., Is elevated noradrenalin an aetiological factor in a number of diseases?, Autonomic and Autacoid Pharmacology (2009) 29:141-156.

### 20. Asthma

Asthma is a common disease that involves airway inflammation and excessive production of mucus by the airways. Norepinephrine, through its second messenger signaling pathways, interacts with various molecules, such as cytokines, to affect asthmatic inflammation. 5-HT_{2C} receptor agonists, which reduce norepinephrine levels, can be used to treat asthma. Fitzgerald P. J., Is elevated noradrenalin an aetiological factor in a number of diseases?, Autonomic and Autacoid Pharmacology (2009) 29:141-156.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Effects of Lorcaserin Administration

A fifty-six-day, double-blind, randomized, placebo-controlled, parallel-group study was conducted to assess the effects of lorcaserin hydrochloride administration to overweight and obese male and female patients. The study was completed according to the guidelines of Good Clinical Practice and was conducted in full compliance with the World Medical Association Declaration of Helsinki and its most recent amendments.

Fifty-seven overweight or obese adult male and female subjects aged 18 to 65 years inclusive were randomized to one of two treatment groups: placebo or lorcaserin hydrochloride (10 mg) twice-a-day (BID) for 56 days. The majority of subjects were female (68.4%) and Caucasian (63.2%). Subjects had a mean (SD) age of 48.7 (12.6) years, ranging between 20 and 64 years, with BMIs ranging between 27.3 and 45.0 kg/m². Patients were required to participate in the Arena Healthy Lifestyle Program®, designed specifically for this study, which included a 600 kcal deficit diet and 30 minutes of moderate exercise per day.

Each subject underwent screening procedures within 28 days or sooner, prior to dosing on Day 1, over 3 screening visits (Days −28, −14, and −7). This was followed by an initial inpatient period of 4 days, a 3-day outpatient period, a second 4-day inpatient period, a second outpatient period over 45 days which included 7 visits, and a final 3-day inpatient period. Fifty-three subjects completed the study. See Table 1.

TABLE 1
Summary of Patient Disposition by Treatment Group

| | Placebo | Lorcaserin | Total |
|---|---|---|---|
| Patients Randomized | 28 | 29 | 57 |
| Completed Study | 25 | 28 | 53 |
| Withdrew | 3 | 1 | 4 |
| Deviation/Non-Compliance with Protocol | 1 | 1 | 2 |
| Other | 1 | 0 | 1 |
| Patient Decision | 1 | 0 | 1 |

Lorcaserin formulation is composed of white tablets containing lorcaserin (active) and excipients (silicified microcrystalline cellulose NF, hydroxypropyl cellulose NF, croscarmellose sodium NF, magnesium stearate NF and Opadry® II white). The placebo is composed of white tablets containing excipients.

All enrolled patients received two oral doses per day (one dose in the morning prior to breakfast and one dose in the evening prior to dinner) of study medication (lorcaserin or placebo) for 56 days. Dosing was performed in a double-blind manner so that neither the patient nor the Investigator knew which treatment had been assigned. Patients were told to take the study medication each morning and evening approximately 60 minutes before breakfast and dinner and were encouraged to take the study medication with an adequate amount of water (8 oz or 240 mL). Patients were asked not to crush, break, chew, or dissolve the tablets.

Patients were required to fast for 10 hours prior to each study visit that requires a blood sample for clinical laboratory tests. Participants were weighed (in duplicate) at each study visit wearing a hospital gown. Efforts were made to schedule study visits prior to 10:00 a.m. to capture the fasted body weight and to reduce the variability in body weight normally observed throughout the day. Weights were measured in kilograms (kg). The scale met NTEP standards, had a precision to the nearest 100 g, and was approved for providing certifiable weights.

The analyses of all efficacy variables used the Modified Intent-to-Treat Population (MITT) population. Patient data were analyzed according to the treatment assigned at randomization, regardless of the treatment received during the course of the trial. Inclusion was dependent on the presence of a baseline measurement, consumption of at least one study dose, and a post-randomization measurement. Patients were instructed to bring their unused study drug with them to each study visit. Compliance was assessed by the number of remaining tablets. Change from baseline or percent change from baseline is defined as the change from Day 1 (randomization) measurement. If the Day 1 measurement was not available, the last non-missing pre-randomization measurement was used as the baseline value, otherwise baseline was considered missing. Baseline assessment as a covariate was used in ANCOVA models in comparing treatment groups. No imputation was applied to any missing values for the efficacy or safety analyses.

### Weight Loss

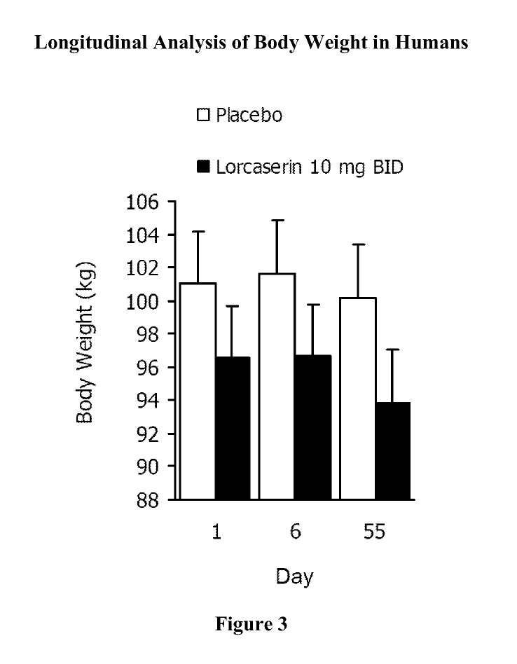

No significant weight loss was observed at 6 days in either the lorcaserin or placebo groups. Significantly greater weight loss was observed at 55 days in the lorcaserin group as compared to placebo. See Table 2 and FIG. 3.

TABLE 2
Bodyweight (kg)

| | |
|---|---|
| Placebo | Lorcaserin |

| Day | N | Mean | N | Mean |
|---|---|---|---|---|
| 1 | 28 | 101.06 | 29 | 96.59 |
| 6 | 27 | 101.63 | 29 | 96.67 |
| 55 | 25 | 100.12 | 27 | 93.84 |

### Urine Catcholamines

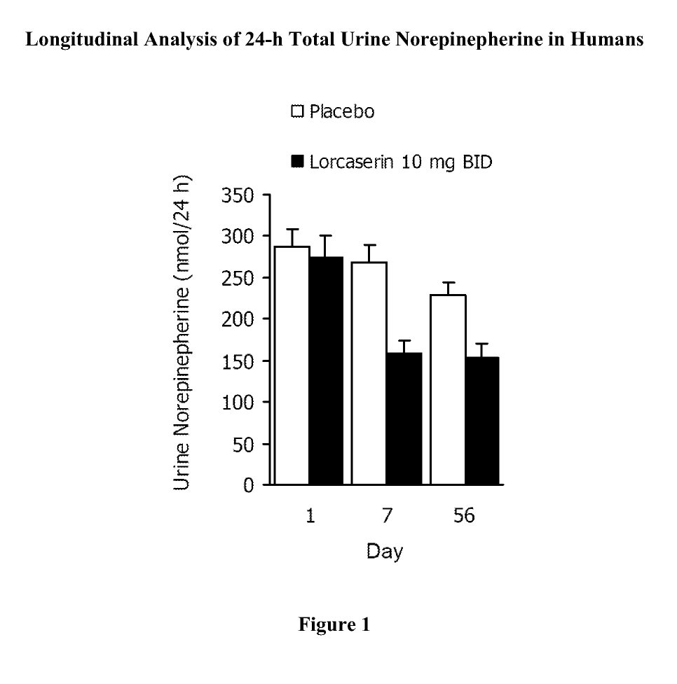

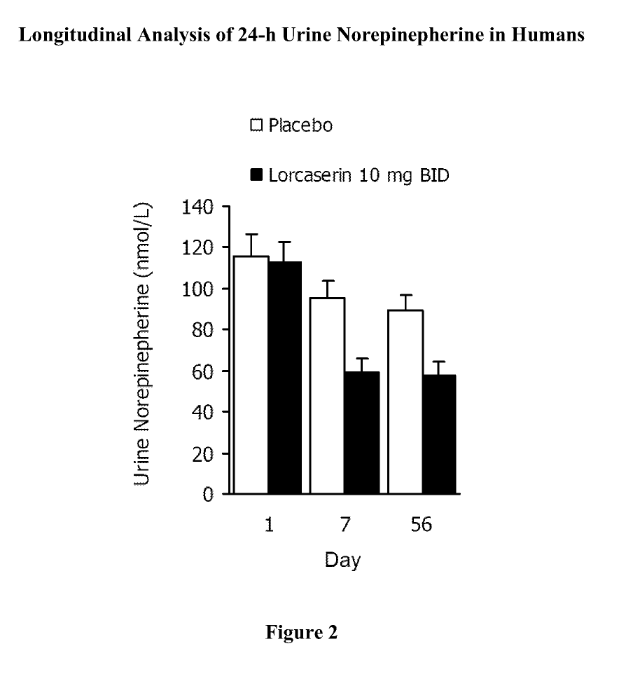

Mean concentrations and mean change from baseline in 24 h urine epinephrine and norepinephrine at Day 7 and Day 56 were assessed. At baseline, 24 h epinephrine and norepinephrine excretion did not differ between lorcaserin and placebo. There was a significant decrease in 24 h norepinephrine excretion in urine after 7 (p<0.0001) and 56 days (p<0.001). See Tables 3 and 4 and FIGS. 1 and 2. Epinephrine was below the limit of quantitation in 44 of 55 available patient samples at Day 7 and 33 of 52 available samples at Day 56. In the small number of evaluable samples, there was no change in 24 h epinephrine excretion after 7 or 56 days of treatment.

TABLE 3
Urine Norepinepherine (nmol/24 h)

| | |
|---|---|
| Placebo | Lorcaserin |

| Day | N | Mean | N | Mean |
|---|---|---|---|---|
| 1 | 27 | 287.16 | 29 | 274.35 |
| 7 | 26 | 269.46 | 29 | 158.20 |
| 56 | 24 | 228.46 | 27 | 152.99 |

TABLE 4
Urine Norepinepherine (nmol/L)

| | |
|---|---|
| Placebo | Lorcaserin |

| Day | N | Mean | N | Mean |
|---|---|---|---|---|
| 1 | 27 | 116.13 | 29 | 112.94 |
| 7 | 26 | 95.67 | 29 | 59.28 |
| 56 | 24 | 89.19 | 27 | 57.34 |

Safety

Lorcaserin 10 mg BID was generally well tolerated and did not increase the incidence of depression related adverse events. There were no early terminations due to AEs. No deaths or serious adverse events occurred in the study.

Headache and dizziness were the only adverse event preferred terms reported by more than 2 patients in the lorcaserin group. Headache was reported by 20 (35.1%) subjects overall and occurred at similar rates between treatment groups: 11 (37.9%) in the lorcaserin group and 9 (32.1%) in the placebo group experienced at least 1 event. The majority of the headaches in both groups were considered to be mild in intensity and either possibly or probably related to study drug. There was 1 episode of severe headache reported. The subject (randomized to placebo) was treated with 500 mg paracetamol PRN and the headache resolved Dizziness was reported by 4 (7%) subjects (3 in the lorcaserin group and 1 in the placebo). All episodes of dizziness were mild or moderate in intensity with a possible relationship to study drug. No deaths occurred during the conduct of the study. No SAEs occurred during the conduct of the study.

A 12-lead ECG was performed at screening (baseline), and during the exit visit (Day 57). There were no apparent treatment-related effects on ECG during the treatment phase. Individual subjects experienced abnormalities at all time points including baseline. Abnormalities were observed in both treatment groups. None of the abnormalities were clinically significant.

Serial assessments of depression were performed during the study at Days −2, +6 and +55 (Beck Depression Inventory-II (BDI-II)). No meaningful differences between the lorcaserin and placebo groups were observed.

### Example 2

### Intracellular IP3 Accumulation Assay

Compounds can be tested for their ability to activate human 5-HT_{2A}, 5-HT_{2B}, and 5-HT_{2C} receptors using, e.g., an IP accumulation assay, which may be performed via the following method.

Human Embryonic Kidney 293 (HEK293) cells are transfected in 15 cm sterile dishes with 16 μg of human 5-HT_{2A}, 5-HT_{2B}, or 5-HT_{2C} receptor cDNA using 25 μL of lipofectamine. (For receptor sequences see e.g., U.S. Pat. No. 6,107,324; U.S. Pat. No. 6,541,209; Schmuck, K. et al., FEBS Letters, 1994, 342, 85-90.) Cells are incubated for 3-4 h at 37° C./5% CO₂ and the transfection medium is removed and replaced with 100 μL of Dulbecco's Modified Eagle Medium (DMEM) (Invitrogen™, Carlsbad Calif.). Cells are then plated onto 100 cm sterile dishes.

The next day, the cells are plated into 96 well PDL microtiter plates at a density of 55K/0.2 mL. After 6 h, the medium is exchanged with [³H]inositol (0.25 μCi/well) in inositol free DMEM and the plates are incubated overnight (37° C./5% CO₂). The next day, the wells are aspirated and DMEM (200 μL) containing a test compound, pargyline (10 μM), and LiCl (10 mM) is added to appropriate wells. Plates are then incubated at 37° C./5% CO₂ for 3 h followed by aspiration and addition of ice-cold stop solution (1 M KOH, 19 mM sodium borate, 3.8 mM EDTA) to each well. The plates are kept on ice for 5-10 min and then the wells are neutralized by the addition of ice-cold neutralization solution (7.5% HCl, 200 μL). The plates are then frozen until further processing is desired.

The lysate is transferred into 1.5 mL Eppendorf tubes and chloroform/methanol (1:2; 1 mL per tube) is added. The solution is vortexed for 15 s and a portion of the upper phase (0.9 mL) is applied to a Biorad AG1-X8™ anion exchange resin column (100-200 mesh) previously washed with water (1:1.25 w/v). The column is then washed with 5 mM myo-inositol (10 mL) and 5 mM sodium borate/60 mM sodium formate (10 mL). The inositol tris phosphates are eluted into scintillation vials containing scintillation cocktail (10 mL) and 0.1 M formic acid/1 M ammonium formate (2 mL) and radioactivity is measured using a scintillation counter.

Those skilled in the art will recognize that various modifications, additions, substitutions, and variations to the illustrative examples set forth herein can be made without departing from the spirit of the invention and are, therefore, considered within the scope of the invention.

1. A method for:
a. reducing a concentration of norepinephrine in an individual; or
b. treating a disorder ameliorated by reducing a concentration of norepinephrine in an individual;
comprising administering to said individual, by said individual or by a caregiver, one or more doses of a medicament comprising a therapeutically effective amount of a 5-HT_{2C} receptor agonist.

2. The method according to claim 1, comprising administering to said individual a plurality of doses of said medicament.

3. The method according to claim 2, comprising administering to said individual one or two doses of said medicament per day for:
at least one week; or
at least one month.

4. The method according to claim 1, wherein said reducing, or amelioration of said disorder occurs:
within 56 days of administering first said dose; or
within 7 days of administering first said dose.

5. The method according to claim 1, wherein said reducing, or amelioration of said disorder is not dependent upon concomitant weight-loss in said individual.

6. The method according to claim 1, wherein said individual does not lose a substantial amount of weight during said reducing or amelioration of said disorder.

7. The method according to claim 1, wherein said individual loses a substantial amount of weight during said reducing or amelioration of said disorder; and wherein said reducing or amelioration of said disorder is greater than the amount of reducing or amelioration expected by said individual or said caregiver solely as a result of said individual loosing said substantial amount of weight.

8. The method according to claim 1, wherein said concentration is:
a urine concentration;
a blood concentration;
a plasma concentration;
a brain concentration; or
a cerebrospinal fluid concentration.

9. The method according to claim 1, wherein said reducing provides reduced concentration of norepinephrine in said individual:
at least about 10% lower than baseline;
at least about 20% lower than baseline;
at least about 30% lower than baseline;
at least about 40% lower than baseline; or
at least about 50% lower than baseline.

10. A method for maintaining said reduced concentration of norepinepherine, according to claim 9, comprising administering to said individual, by said individual or by a caregiver, one or more doses of a medicament comprising a therapeutically effective amount of a 5-HT_{2C} receptor agonist.

11. The method according to claim 9, wherein said reduced concentration is:
a urine concentration;
a blood concentration;
a plasma concentration;
a brain concentration; or
a cerebrospinal fluid concentration.

12. A method for manufacturing a medicament for reducing a concentration of norepinephrine in an individual, or for treating a disorder ameliorated by reducing a concentration of norepinephrine in an individual comprising admixing a 5-HT₂c receptor agonist with at least one pharmaceutically acceptable excipient; wherein said 5-HT₂c receptor agonist has been administered to a mammal in whom a norepinephrine concentration has been measured.

13. A method for manufacturing a medicament for reducing a concentration of norepinephrine in an individual, or for treating a disorder ameliorated by reducing a concentration of norepinephrine in an individual comprising:
a. measuring a norepinephrine concentration in a mammal; and
b. admixing a 5-HT_{2C} receptor agonist with at least one pharmaceutically acceptable excipient;
wherein said 5-HT_{2C} receptor agonist has been administered to said mammal.

14. A method for manufacturing a medicament for reducing a concentration of norepinephrine in an individual, or for treating a disorder ameliorated by reducing a concentration of norepinephrine in an individual comprising:
a. administering a 5-HT_{2C} receptor agonist to a mammal;
b. measuring a norepinephrine concentration in said mammal; and
c. admixing said 5-HT_{2C} receptor agonist with at least one pharmaceutically acceptable excipient.

15. Method for manufacturing a medicament for reducing a concentration of norepinephrine in an individual, or for treating a disorder ameliorated by reducing a concentration of norepinephrine in an individual comprising resynthesizing a 5-HT_{2C} receptor agonist; wherein said 5-HT_{2C} receptor agonist has been administered to a mammal in whom a norepinephrine concentration has been measured.

16. A method for manufacturing a medicament for reducing a concentration of norepinephrine in an individual, or for treating a disorder ameliorated by reducing a concentration of norepinephrine in an individual comprising:
a. measuring a norepinephrine concentration in a mammal; and
b. resynthesizing a 5-HT_{2C} receptor agonist;
wherein said 5-HT_{2C} receptor agonist has been administered to said mammal.

17. A method for manufacturing a medicament for reducing a concentration of norepinephrine in an individual, or for treating a disorder ameliorated by reducing a concentration of norepinephrine in an individual comprising:
a. administering a 5-HT_{2C} receptor agonist to a mammal;
b. measuring a norepinephrine concentration in said mammal; and
c. resynthesizing said 5-HT_{2C} receptor agonist.

18. The method according to claim 1, wherein said disorder is selected from: hypernorepinephrinemia, cardiomyopathy, cardiac hypertrophy, cardiomyocyte hypertrophy in post-myocardial infarction remodeling, elevated heart rate, vasoconstriction, acute pulmonary vasoconstriction, hypertension, heart failure, cardiac dysfunction after stroke, cardiac arrhythmia, metabolic syndrome, abnormal lipid metabolism, hyperthermia, Cushing syndrome, pheochromocytoma, epilepsy, obstructive sleep apnea, insomnia, glaucoma, osteoarthritis, rheumatoid arthritis, and asthma.

19. The method according to claim 1, wherein said individual is a human.

20. The method according to claim 1, wherein said 5-HT_{2C} receptor agonist has an EC₅₀ of:
less than about 10 μM at the 5-HT₂c receptor;
less than about 1 μM at the 5-HT₂c receptor; or
less than about 100 nM at the 5-HT₂c receptor.

21. The method according to claim 1, wherein said 5-HT_{2C} receptor agonist is a selective 5-HT_{2C} receptor agonist.

22. The method according to claim 21, wherein the ratio of the EC₅₀ of said selective 5-HT_{2C} receptor agonist at the 5-HT_{2C} receptor to the EC₅₀ of said selective 5-HT_{2C} receptor agonist at the 5-HT_{2A} receptor is:
at least about 10:1;
at least about 100:1; or
at least about 1000:1.

23. The method according to claim 21, wherein the ratio of the EC₅₀ of said selective 5-HT_{2C} receptor agonist at the 5-HT_{2C} receptor to the EC₅₀ of said selective 5-HT_{2C} receptor agonist at the 5-HT_{2B} receptor is:
at least about 10:1;
at least about 100:1; or
at least about 1000:1.

24. The method according to claim 21, wherein said selective 5-HT_{2C} receptor agonist is a partial agonist, an antagonist, an inverse agonist, or a neutral antagonist of the 5-HT_{2A} receptor.

25. The method according to claim 21, wherein said selective 5-HT_{2C} receptor agonist is a partial agonist, an antagonist, an inverse agonist, or a neutral antagonist of the 5-HT_{2B} receptor.

26. The method according to claim 1, wherein said 5-HT_{2C} receptor agonist is a small molecule.

27. The method according to claim 1, wherein said 5-HT_{2C} receptor agonist is orally-bioavailable.

28. The method according to claim 27 wherein said selective 5-HT_{2C} receptor agonist is selected from 8-chloro-1-methyl-2,3,4,5-tetrahydro-1H-3-benzazepine, and pharmaceutically acceptable salts, solvates, and hydrates thereof.

29. The method according to claim 27, wherein said selective 5-HT_{2C} receptor agonist is (R)-8-chloro-1-methyl-2,3,4,5-tetrahydro-1H-3-benzazepine hydrochloride salt hemihydrate.

30.-50. (canceled)
